(19) United States
(12) Patent Application Publication
(10) Pub. No.: US 2007/0264354 A1
(43) Pub. Date: Nov. 15, 2007

(54) TREATMENT OF A WOUND WITH A VASODILATOR

(76) Inventor: Richard M. Herman, Scottsdale, AZ (US)

Correspondence Address: LEWIS AND ROCA LLP, 40 N. CENTRAL AVENUE, Suite 1900, PHOENIX, AZ, 85004-4429, US

(21) Appl. No.: 11/745,178

(22) Filed: May 7, 2007

Related U.S. Application Data
(60) Provisional application No. 60/746,849, filed on May 9, 2006.
(60) Provisional application No. 60/887,756, filed on Feb. 1, 2007.

(51) Int. Cl. A61K 31/385; A61K 31/195; A61K 33/00

(52) U.S. Cl. 424/608; 514/12; 514/440; 514/565

(57) ABSTRACT

A wound treatment administers a vasodilator by: (a) active transdermal treatment such as: by direct perfusion of the wound with the vasodilator, by an intradermal injection of the vasodilator about the wound and its surrounding skin area, by iontophoresis about the wound and its surrounding skin area with the vasodilator, by microdialysis of the wound with the vasodilator using one or more probe situated about the wound and its surrounding skin area; and/or by (b) passive transdermal treatment such as: by transcutaneous electrical stimulation (TENS) about the wound and its surrounding skin area while administering the vasodilator, or by a combination of the foregoing. The vasodilator can be calcitonin gene-related peptide (CGRP) with or without vasoactive intestinal polypeptide (VIP), nitric oxide (NO), nerve growth factor (NGF), or a combinations of the forgoing. Blood flow proximal the wound can be monitored during the treatment to derive a chemical/dose relationship of the vasodilator being administered in the treatment.

### CROSS REFERENCES TO RELATED APPLICATIONS

This application claims priority to U.S. Provisional Application Ser. No. 60/746,849, titled “Treatment of A Wound With A Vasodilator”, filed on May 9, 2006, to Richard M. Herman, and to U.S. Provisional Application Ser. No. 60/887,756, titled “Treatment of A Wound With A Vasodilator”, filed on Feb. 1, 2007, to Richard M. Herman, both of which are incorporated herein by reference

### TECHNICAL FIELD

The present invention relates generally to wound treatment, and is more particularly related to the treatment of a wound with a vasodilator such as calcitonin gene-related peptide (CGRP).

### BACKGROUND

Microvascular dysfunction is a major cause of impaired wound healing and occurs in both rodents and humans with diabetes, venous stasis, ischemic skin flaps, aging, obesity and spinal cord injury (Kjartannson et al, 1987, 1988; Khalil et al, 1994; Jernberg and Dalsgaard, 1993; Carr et al, 1993; Gherardini et al, 1998a,b; Parkhouse and LeDuesne, 1988; Lundeberg, 1993; Ardron et al, 1991; Appenzeller and McAndrews, 1966). In almost all conditions of microvascular failure, there is functional and anatomical impairment of a group of primary afferent unmyelinated fibers in the skin called “nociceptors, C-fibers” (See “Study Section”). These C-fibers release neuropeptides, particularly calcitonin gene-related peptide (CGRP), the most abundant peptide in human skin (Holzer, 1998; Wallengren, 1997) which controls vascular tone of the microvessels and exhibits both trophic and immunomodulatory influences on tissue.

When denervation of C-fibers occur and release of neuropeptides is reduced, as Richards et al (1997) point out, wounds can occur which are associated with cellular changes with altered chemotaxis. Moreover, neuropeptide depletion is a common histopathological end-point in various clinical conditions such as Raynaud's Phenomenon, diabetes, obesity, aging, and skin-flaps, all of which exhibit microcirculatory impairment. Under these conditions, CGRP (also vasoactive intestinal polypeptide (VIP) and substance P (SP)) immunoreactivity is markedly decreased (Levy et al, 1989; Schmelz et al, 1997). Depletion of neuropeptides and nitric oxide (NO) lead to depressed response to skin injury and, hence, reduction in eliciting an inflammatory (vasodilator) reaction as well as diminished trophic and immunomodulatory functions.

It would be an advance in the art to provide an effective treatment for the healing of such wounds and for the prevention of the occurrence of such wounds.

### SUMMARY

A vasodilator is administered to treat wound. The vasodilator may be administered by: (a) active transdermal treatment such as by direct perfusion of the wound with the vasodilator, by an intradermal injection of the vasodilator about the wound and its surrounding skin area, by iontophoresis about the wound and its surrounding skin area with the vasodilator, by microdialysis of the wound with the vasodilator using one or more probe situated about the wound and its surrounding skin area; and/or by (b) passive transdermal treatment such as by transcutaneous electrical stimulation about the wound and its surrounding skin area while administering the vasodilator, and by a combination of the foregoing treatments.

A variety of vasodilators can be administered, including calcitonin gene-related peptide (CGRP), vasoactive intestinal polypeptide (VIP), nitric oxide (NO), nerve growth factor (NGF), and combinations of the forgoing.

### BRIEF DESCRIPTION OF THE DRAWINGS

A more complete understanding of the implementations may be had by reference to the following detailed description when taken in conjunction with the accompanying drawings wherein:

FIG. 1 is a perspective view of an exemplary perivascular C-Fiber complex;

FIG. 2 is a perspective view of exemplary implementations of methods for wound treatment that can be performed upon a wound located on a human foot; and

FIG. 3 is a perspective view of exemplary implementations of methods for wound treatment that can be performed upon a wound located on a human hand.

FIG. 4, referred to in the Study Section below, show a graph to demonstrate that during a heat-ramp test, heat-induced flux in the capsaicin-desensitized subjects (Cap-D) decreased significantly under the thermode (direct flow) and also outside the thermode (indirect flow).

FIG. 5, referred to in the Study Section below, shows a graph to demonstrate that the normal (with vehicle) flare or neurogenic vasodilatory response increases with increasing doses of cathodal current. Following capsaicin-desensitization, the dose-response curve is abolished, suggesting suppression of the nerves mediating the axon-reflex.

FIG. 6, referred to in the Study Section below, shows a graph to demonstrate that both direct and indirect flow sites showed a capsaicin-induced peak flow (flux) that was significantly lower in obese subjects compared to normals.

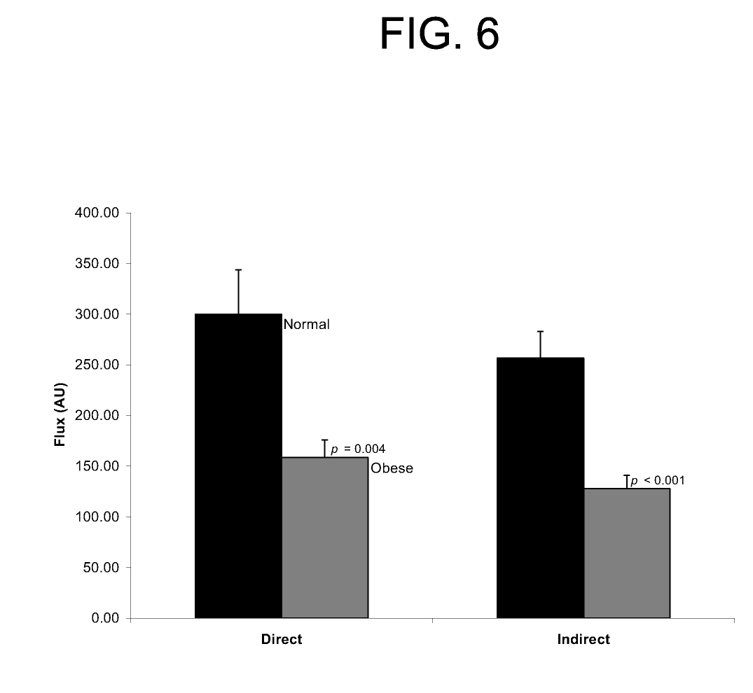

FIG. 7, referred to in the Study Section below, shows a graph to demonstrate that the latency to capsaicin-induced direct and indirect (flare) blood flow was significantly prolonged in the obese subjects.

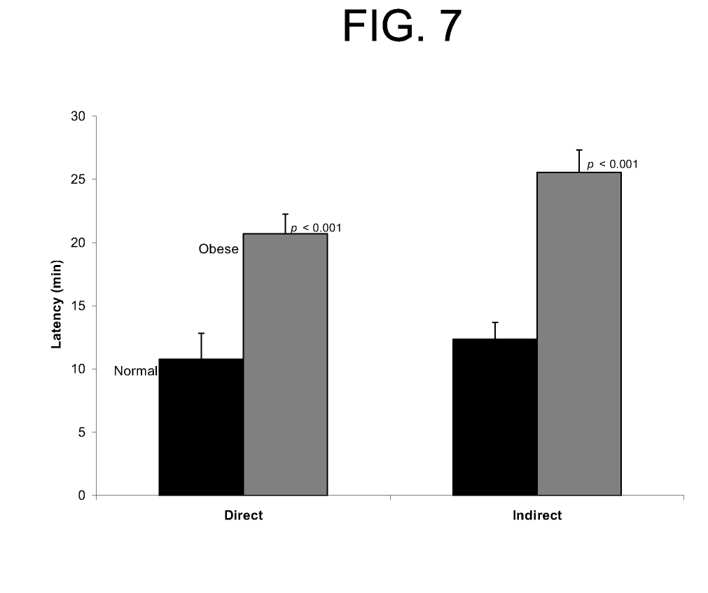

FIG. 8, referred to in the Study Section below, shows a graph to demonstrate that a significant (p<0.001) negative correlation exists between flux (indirect flow) and latency with a correlation coefficient of R²=0.401 among the obese subjects.

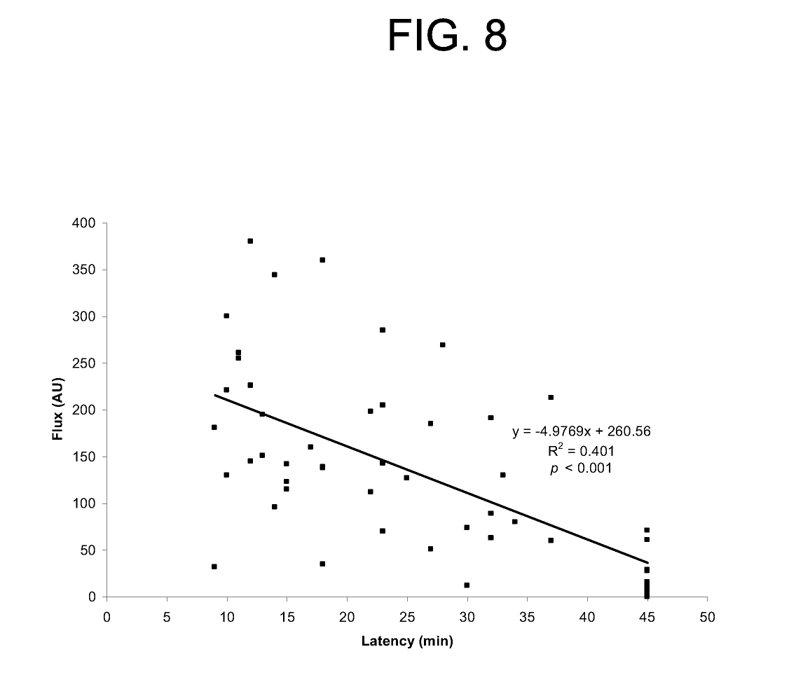

FIG. 9, referred to in the Study Section below, shows a graph to demonstrate that, prior to capsaicin application (Pre-Cap), the HPT to a step-increase in temperature was significantly raised in the obese group. Following capsaicin (Post-Cap), the HPT was significantly (p<0.001) lowered in both groups, indicating primary hyperalgesia.

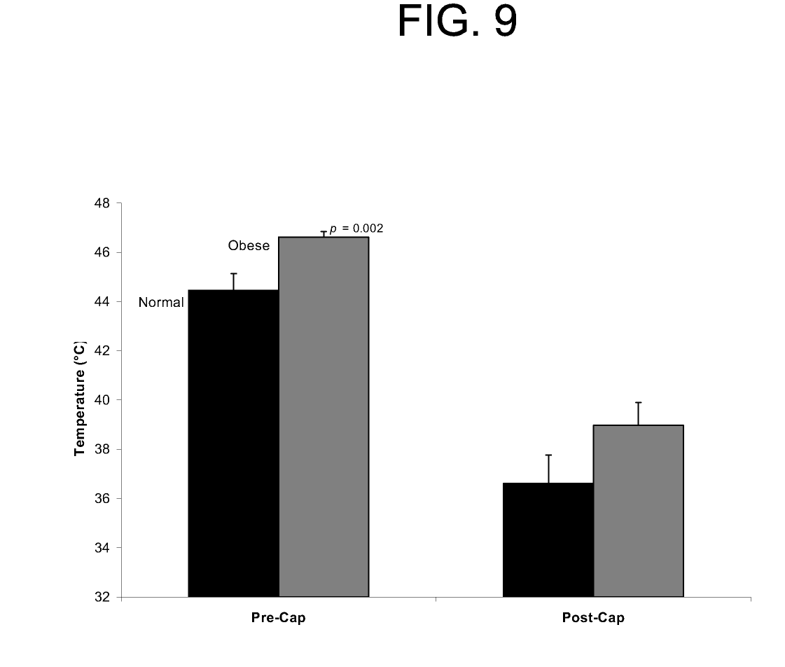

FIG. 10, referred to in the Study Section below, shows graphs to demonstrate that, blood flow responses to a ramp increase in temperature from 32° C.-44° C. Note the significant reduction in the flare (neurogenic vasodilatory) response whereas the responses under the thermode were insignificant.

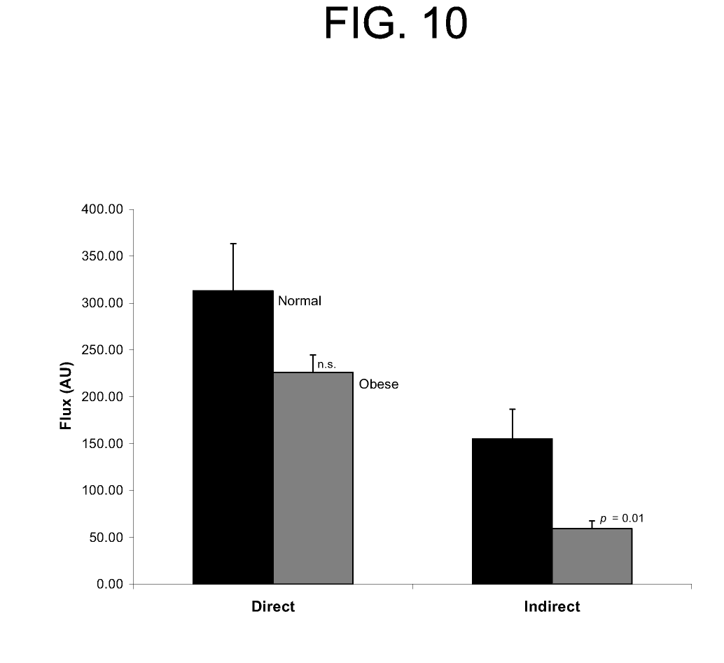

FIG. 11, referred to in the Study Section below, shows a graph to demonstrate that the conduct of the dose-response curve was similar to that described in FIG. 1B in that under normal conditions, blood flow increased gradually with the magnitude of the dose and under conditions of obesity (like capsaicin-desensitization) the dose-response curve was markedly diminished.

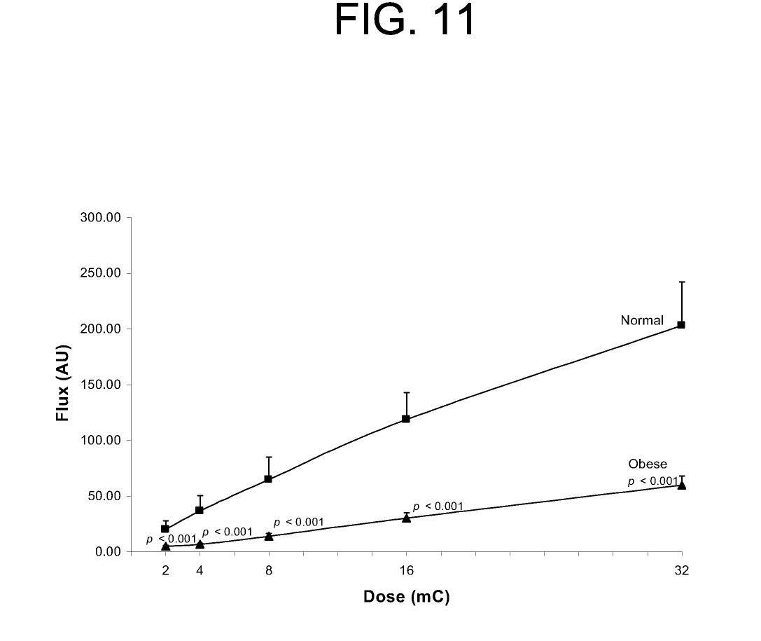

### DESCRIPTION

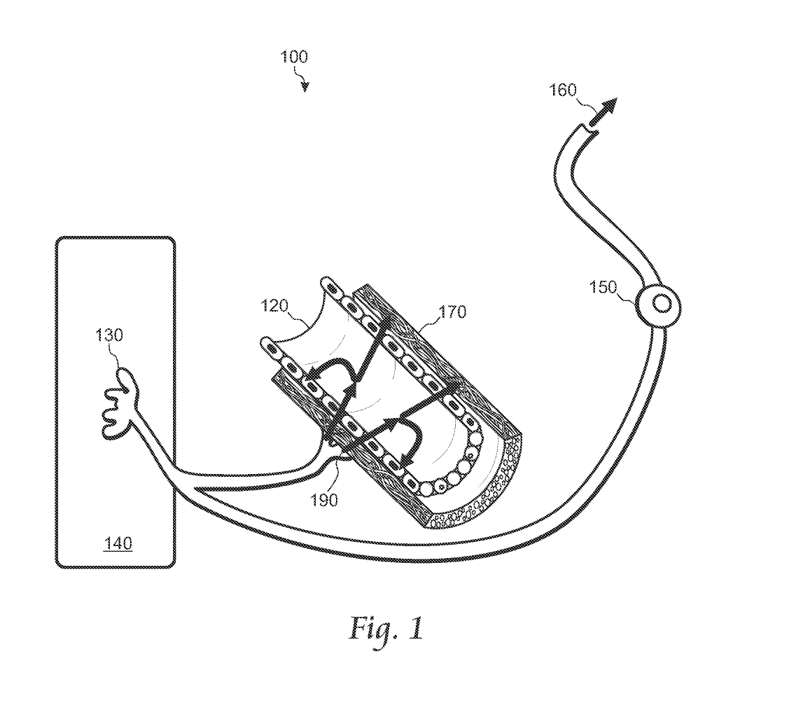

FIGS. 1-3 and this description propose a paradigm for wound healing and for the prevention of wound occurence.

1. Perivascular C-Fiber Complex.

FIG. 1 depicts an example of a perivascular C-Fiber complex 100. Shown in FIG. 1 is a subset of a nerve system in the periphery (e.g.; in the skin 140) called C-Fibers 100 in that there are not any myelin or protective coating around it or protective coating. As illustrated by FIG. 1, the endothelium is seen at 120, the polymodal receptor at 130, the skin at 140, the cell body at 150, the direction to the central nervous system (CNS) at 160, the vascular smooth muscle at 170, CGRP at 190, and NO at 190. The depicted fibers go to the CNS from left to right as seen at 160 in FIG. 1 to the brain, for instance to convey pain sensation. It also goes to the local blood vessels in the skin and it releases certain chemicals like CGRP, Substance P and NO. It also affects the endothelium to produce more NO so that there are two vasodilators that work together, i.e.; CGRP and NO at 190.

If the C-Fiber 100 seen in FIG. 1 is impaired, the blood vessel will constrict, the tissue will be poorly perfused, and the subject will be liable to breakdown such that it cannot handle any injury to the skin. If there is an injury to the skin without vessel dilation, the skin will break down and there will be an ulcer or a wound.

2. Wound Treatment

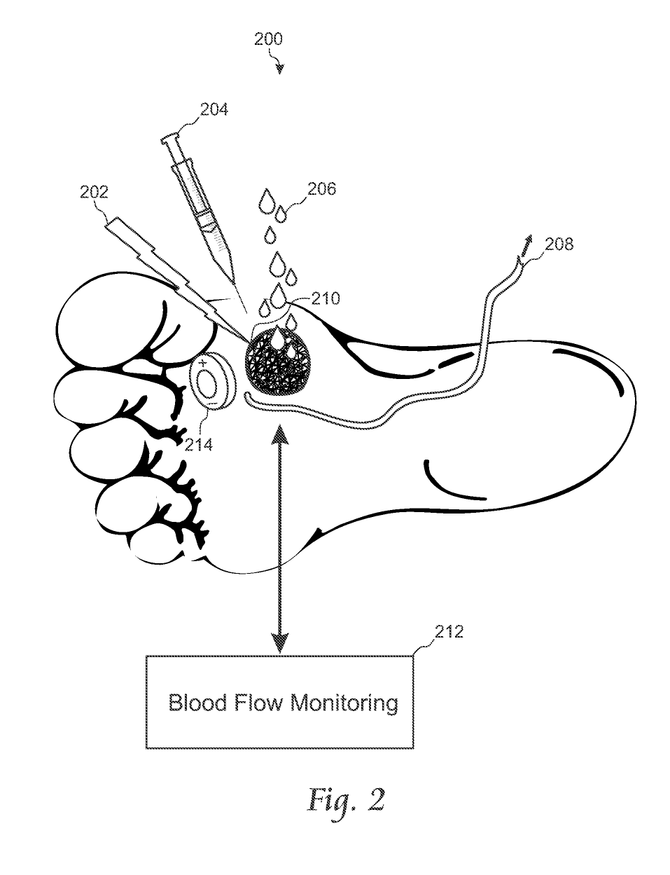

FIG. 2 depicts a wound located on a human foot, in a perspective view, in an environment for administration of any of five (5) wound treatments, or a combination thereof.

The first method is a direct infusion 206 of the wound by the administration of a droplet of a vasodilator (e.g.; a neuropeptide) on top of the wound, as shown in FIG. 2. Also illustrated in FIG. 2 is an intradermal infusion through a syringe 204 into the skin around the wound.

FIG. 2 depicts a method involving iontophoresis 214, which is an electrical way of transferring a chemical through the wound and over the skin as well. A microdialysis method 208 of wound treatment is also seen in FIG. 2. The microdialysis method can employ catheters that are put under the skin. While under the skin, the catheters drip out a vasodilator (e.g.; a peptide such as CGRP) over a period of time.

An electrical stimulation method 202, including but not limited to Transcutaneous electrical stimulation (TENS), is also shown in FIG. 2. In this method, the blood vessels are dilated. Also shown in FIG. 2. is an iontophoresis method 214 of wound treatment. Like the electrical stimulation method, the iontophoresis method has an electric effect. A passive transdermal method 210 is also contemplated.

Given the foregoing, the five (5) Treatment Methods depicted in FIG. 2 relate to:
(a) active transdermal treatment such as:
   A. Direct perfusion of the wound 206;
   B. Intradermal injections about the wound/skin area 204;
   C. Iontophoresis about the wound/skin area 214;
   D. Microdialysis probes about the wound/skin area 208; and/or
(b) passive transdermal treatment such as:
   E. Transcutaneous electrical stimulation (TENS) about the wound/skin area 202, 210
      where combinations of the foregoing treatments A-E may be used to treat wounds.

For each of the foregoing five (5) Treatment Methods depicted in FIG. 2, it is advantageous to monitor blood flow 212 to ascertain whether a desirable physiological effect is being achieved, for instance dilation. By way of example, but not by way of limitation, one method of measuring blood flow is by the use of laser Doppler flowmetry (LDF) and/or laser Doppler perfusion imagery (LDPI). In all such applications, the blood flow caused by the chemical/device can be monitored so as to determine the dose/response relationship to augment perfusion.

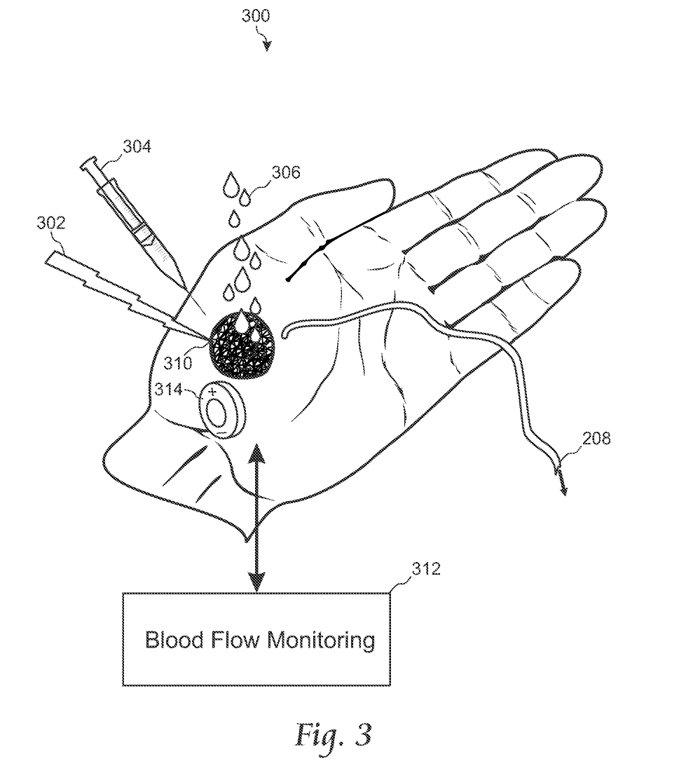

FIG. 3 depicts a wound located on a human hand, in a perspective view, in an environment for administration of any of five (5) wound treatments, or a combination thereof, as described above with respect to FIG. 2, namely:

(a) active transdermal treatment:
A. Direct perfusion of the wound 306;
B. Intradermal injections about the wound/skin area 304;
C. Iontophoresis about the wound/skin area 314;
D. Microdialysis probes about the wound/skin area 308; and/or

(b) passive transdermal treatment:
E. Transcutaneous electrical stimulation (TENS) about the wound/skin area 302, 310

Blood flow is also monitored (312) as shown in FIG. 3.

When denervation of C-fibers occurs and release of neuropeptides is reduced, as mentioned in the Background (above), wounds can occur which are associated with cellular changes with altered chemotaxis. The application of calcitonin gene-related peptide (CGRP) with or without other molecules associated with C-fibers will promote wound healing under various methods of administration, where such other molecules include but are not limited to vasoactive intestinal polypeptide (VIP), nitric oxide (NO), and nerve growth factor (NGF). These methods of administration will be applicable to those wounds associated with cellular changes with altered chemotaxis that result when there is a denervation of C-fibers accompanied by a reduction in the release of neuropeptides. With each such method administration, the doses can be adjusted by monitoring blood flow.

In each of the foregoing five (5) Treatment Methods A-E, and their combinations, a vasodilator is administered. Preferably, the vasodilator will be CGRP. Alternatively, VIP, NO), or NGF could also be administered, including administrations having combinations thereof, and where such administrations may also include CGRP.

Injured tissue, particularly associated with ischemia, reveals up-regulation to CGRP and possibly to NO. In any case, CGRP should influence NO expression given sufficient NO Synthase (NOS) and reduced oxidative stress. Thus, the formula proposed herein for treatment emphasizes CGRP being delivered by various means to produce its vasodilator, trophic and immune effects on a wound considered “simple”, i.e., not infected. If there is a need for a more vigorous approach, there is envisioned the use of other peptides such as VIP and a NO-donor such as sodium nitroprusside by the Treatment Methods A-E, or combinations thereof. Further supplementation may be required which would include systemic L-Arginine and an anti-oxidant, e.g., alpha-lipoic acid.

In support of the forgoing proposed treatments, the following narrative provides a perspective of relevant research to demonstrate a long felt need for such treatments.

3. Epidemiology.

The morbidity and mortality from Raynaud's Phenomenon, venous stasis ulcers, diabetic foot ulcer or pressure ulcers (sacral, gluteal or trochanteric) presents serious health care problems. It has been calculated that the total prevalence of the latter 3 types of ulcers is between 3 and 6 million (Brem et al, 2000). These physiologically impaired and slow-to-heal wounds place a great burden on the health system, with costs considerably greater than 10 billion dollars/year. These costs do not include the pain and suffering incurred by the patient who may enter the hospital (or nursing home) for a medical disease and who may leave with a sacral ulcer. Among 16 million patients with diabetes in the U.S., an estimated 12% had a history of foot ulcer between the years 2000-2002 (Centers for Disease Control); approximately 60% of all lower extremity amputations occur among persons with diabetes and, of these, approximately 85% are preceded by a foot ulcer (see also Pecoraro et al, 1990). Limb amputation in patients with diabetes associated with an increased risk for further amputation, which has a 5-year mortality rate of 39%-68% (Reiber et al, 1995). The direct costs for an amputation range from $20,000-$60,000. When the cost of failed vascular reconstruction and rehabilitation-as well as lost productivity within society—is accounted for, the total costs to society exceed financial analysis. Nevertheless, the costs to society of caring with wounds include the market for wound-care products which are estimated to exceed $7 billion annually (Langley-Hawthorne, 2004). A recent accounting of homehealth services provided by 7 million patients found that one-third are being seen for the treatment of persistent wounds at a cost of more than $42 billion yearly (Doughty, 2004). Focusing just on one type of chronic wound, the pressure ulcer, total hospital charges were 5.3 times greater than the charges for all other hospitalized patients and the mean length of stay of patients with pressure ulcers was 4.5 times greater; this is in the face of a prevalence of pressure ulcers in the U.S. estimated to be 1.3-3 million (Lyder, 2003) Again, these numbers give only a sense of the magnitude and direct costs of treating wounds, both acute and chronic, but do not account for indirect costs such as lost lives and limbs and the attendant decrease in productivity as well.

Pressure ulcers also cause pain, lost productivity, and huge expenditures (O'Brien et al, 1999). Pressure ulcers are also common in the elderly population, in patients who are bedridden, in patients with SCI and after major orthopedic reconstruction. The prevalence of pressure ulcers (stage II and greater) is estimated to be up to 17% in hospitalized patients (Allman, 1989), at least 10% in nursing homes and 20-30% in patients with SCI. The healing rate for stage II ulcers that existed only to the dermis has been as low as 25% at 6 months. The definition of a stage II ulcer is: partial-thickness skin loss involving the epidermis or dermis of both. The ulcer is superficial and presents clinically as an abrasion, blister, or shallow crater. In practical terms, it is not uncommon for patients to be discharged with large unhealed ulcers. With a burgeoning aging population, it is imperative to develop new strategies for treatment and prevention. Healing of acute wounds (e.g., surgical) occurs sequentially in a timely fashion and without definitive intervention. Usually, platelets enter the wound and secrete growth factors that subsequently recruit macrophages into the wound. Macrophages also release growth factors (Nathan, 1987) that cause endothelial cells to migrate and proliferate in the wound, thereby stimulating angiogenesis and the fibroblasts to synthesize collagen (Brem and Folkman, 1994). The orderly and timely reparative process characteristic of acute wounds results in a sustained restoration of anatomic and functional integrity (Lazarus et al, 1994).

A chronic wound is characterized by failure to heal in a timely and orderly process, compromising functional and anatomical integrity. Any approach, to be successful, should be relatively simple to administer, with low to minimal side-effects, and should improve wound-healing when compared to non-treated wounds. Healing of chronic wounds chiefly requires concurrent consideration of the inflammatory environment of the wound, persistent infection of the wound, and possible regional ischemia (Isenberg et al, 2005). Addressing such multiple impediments to healing will be necessary to achieve closure of chronic wounds. In any treatment, one must be aware of the HAWTHORNE effect, i.e., the interest itself and the related increase in wound care often result in improved wound healing unrelated to the “magic” agent being investigated.

Diabetic foot ulcers and pressure ulcers are all chronic wounds, and intervention is always necessary to prevent their further progression. Brem et al (2000) point out that higher debridement rates (usually in stage III-IV) correlate with higher healing rates. Thus, at least two debridements will be necessary prior to the application of neuropeptide therapy. The wound should be as “simple” as possible. Debriding any wound to the level at which scar and infection are no longer present (even to bone) has proved to be safe and therapeutic. Viable tissue should not be excised. The wound margins should not extend more than 1-2 mm.

4. Role of Innervation in Wound Pathophysiology.

McGrouther and Ahmad (1998) and Khalil and Helme (1996) point out that skin is capable of mounting a marked inflammatory reaction, an essential component of wound healing. It has long been known that the response of skin to injury involves a cutaneous inflammatory reaction (Lewis, 1927). This is thought to be mediated by a functional relationship between neural and vascular components in the skin (Burnstock and Ralevic, 1994). Evidence suggests that injury causes release of neuropeptides from sensory nerve endings in the skin (Holzer, 1995). These neuropeptides are thought to initiate a neurogenic inflammatory response (principally neurogenic vasodilatation in humans; Weidner et al, 2000) by acting on the cutaneous microvasculature. It has been demonstrated that a suction blister lesion causes an immediate increase in nerve immunoreactivity both in the dermis and epidermis (Gu et al, 1994). The nerves located in the epidermis have previously been suggested to be sensory in nature, that is capsaicin-sensitive nerves or C-fibers (see “Study Section”). That CGRP is found in some amount in the epidermis and dermis suggests at least that some of the nerves identified using a pan-neuronal marker, PGP 9.5 are C-fibers (see also Dalsgard et al, 1989; Wallengren and Chen, 2001). According to accepted models of neurogenic inflammation (including neurogenic vasodilatation; flare; Burnstock and Ralevic, 1994; Foreman, 1987), it is expected that the observed neural change in C-fibers, correlates with the release of sensory neuropeptides from sensory nerve endings. However, this pro-inflammatory effect of C-fibers (e.g., neurogenic vasodilatation) contrasts with an anti-inflammatory action of C-fibers (Raud et al, 1991), i.e., endogenous or exogenous action of CGRP mediates wound healing by both mechanisms.

Wound repair is a dynamic and highly complex process that is dependent on a number of general factors including nutritional status and concurrent morbidity, as well as specific factors that affect the microenvironment at the cellular and neurovascular levels. Our primary interest is in the role of the neurovascular system, specifically the role of small sensory fibers known as C-fibers. An intact nociceptor system of these primary afferent sensory nerves is an essential pre-requisite for successful wound healing as a consequence of the neuropeptides that they release on neighboring tissues such as microvessels (Richards et al, 1997, 1999). Key animal experiments demonstrated that “intentional” denervation of C-fibers by pre-treatment of the skin with capsaicin (CAP) with presumed loss of CGRP and other peptides (see also Raud et al, 1991) decreased survival of ischemic flaps and impaired healing of blister ulcers (Kjartansson et al, 1987; Carr et al, 1993). This latter notion was supported by a number of reports which showed that calcitonin gene-related peptide (CGRP) and substance P (SP) have vasodilator, trophic, and neuroimmunomodulatory functions. For example, they have growth-promoting properties. Trophic influences may be mediated by their vasodilator properties and the ability to stimulate the proliferation of endothelial cells, arterial smooth muscles and skin fibroblasts. A recent document by Richards et al (1999) has proposed that in the presence of small fiber (C-fiber) neuropathy with a presumed loss of neuropeptides, there are reductions in monocyte, macrophage and T lymphocyte counts. Macrophages are important in wound healing and fibrosis because of their ability to secrete cytokines, particularly transforming growth factor-β (TGF-β). A reduced monocyte and macrophage chemotaxis may be responsible for delayed wound contraction. Sensory neuropeptides such as CGRP and SP, contained in and secreted by C-fibers are strongly chemotactic for inflammatory cells, including macrophages and T-lymphocytes in skin wounds and can be used endogenously and exogenously to promote wound healing.

Richards et al (1999) went into further detail and summarized the work to date. Wound healing is a complicated process consisting of 3 main phases: inflammation, connective tissue deposition and remodeling. A breach in tissue continuity triggers clotting and complement cascades which in turn initiates the chemotaxis of inflammatory cells into the wound (these cells appear to play a crucial part in coordinating wound healing by the secretion of various cytokines like platelet-derived growth factor and TGF-β-the latter is secreted by macrophages and is known to be a strong chemoattractant for fibroblasts, recruiting neutrophils, macrophages and lymphocytes. Inflammatory cells including macrophages and lymphocytes therefore form a crucial step in the process of wound healing and fibrosis. These cells together with platelets coordinate wound healing and influence the activity of the fibroblasts by secreting growth factors including TGF-β). The recruitment and activity of these inflammatory cells is thus central to wound healing.

Evidence suggests that neural input may play an important part in modulating wound healing. Substance P (SP) is a tachykinin neuropeptide that is present in small-diameter primary afferent nerves which belong to the C-fiber group. SP has been shown to influence inflammatory responses by stimulating mononuclear and polymorphonuclear leukocyte chemotaxis. It is also able to prime human neutrophil activation and its intradermal (id) injection into skin has been shown to induce neutrophil infiltration. SP receptors have been reported on mast cells, polymorphonuclear leukocytes and macrophages. CGRP increases cellular infiltration into tissue after injection and is known to be chemotactic for human T-lymphocytes and neutrophils. Interestingly, the phenotypic expression of SP and CGRP in primary sensory neurons is regulated by nerve growth factor (NGF). As early as 1980. Li et al (1980) showed that NGF accelerated wound contraction. Lawman et al (1985) in 1985 showed that NGF injected into subdermal air sacs in mice significantly increased leukocyte chemotaxis.

In the Lawman et al (1985) study in 1985, macrophage and T-lymphocyte counts were significantly reduced at 4 days in denervated wounds. This decrease is most like due to a reduced production of cytokines which may in turn lead to decreased contraction seen in these wounds. Their results would indicate that denervation may slow down the rate of wound healing by reducing the inflammatory infiltrate. There is increasing evidence that the nervous system has regulatory actions on the immune system (neuroimmunomodulation). Neuropeptides contained and secreted by nerves have been shown to have specific binding sites and effects on lymphocytes and mononuclear cells. Interestingly these peptides are widely present in cutaneous nerve terminals distributed below and within the epidermis and around blood vessels and glands (Wallengren and Chen, 2002). It is tempting to speculate that neuropeptides may have a part to play in the control of the immune system and, in particular, cellular chemotaxis.

Schoolmen (1998) also expanded upon the role of the nervous system in skin integrity and wounds in a review article. They claim that certain skin diseases such as psoriasis and atopic dermatitis have a neurogenic component. Neuropeptides released by sensory nerves that innervate the skin and often contact epidermal and dermal cells can directly modulate functions of keratinocytes, Langheran cells (LC), mast cells, dermal microvascular endothelial cells, vascular smooth muscle cells and infiltrating immune cells. Many of these peptides—e.g., CGRP, SP, VIP have been reported to effectively modulate skin and immune cell functions such as cell proliferation, cytokine production or antigen presentation under physiological or pathophysiological conditions. Expression and regulation of their corresponding receptors that are expressed on a variety of skin cells as well as the presence of neuropeptide-specific peptidases such as neutral endopeptidase or angiotensin-converting enzyme (ACE) determine the final biological response mediated by these peptides on the target cell or tissue. Likewise, skin cells like keratinocytes or fibroblasts are a source of neurotrophins such as nerve growth factor that are required not only for survival and regeneration of sensory neurons but also to control the responsiveness of these neurons to external stimuli. Therefore, neuropeptides, neuropeptide receptors, neuropeptide-degrading enzymes and neurotrophins participate in a complex, interdependent network of mediators that modulate skin inflammation, wound healing and the skin immune system.

5. Calcitonin Gene-Related Peptide.

Calcitonin Gene-Related Peptide (CGRP) (Wallengren, 1997; Goodman & Iversen, 1986; Wimalawansa, 1996) is a 37 amino acid peptide, which coexists with Substance P (SP) and Neurokinin A (NKA) in sensory nerve cell bodies, i.e., the dorsal root ganglions (DRGs). CGRP is the most abundant of all neuropeptides in human skin and is co-localized with SP. CGRP is a potent endogenous vasodilator of the microcirculation. Together with the observations that CGRP circulates in normal subjects at relatively high concentrations (approximately 25 pmol) and that CGRP is present in perivascular tissues, Struthers et al, 1986 reported the possible role for CGRP in controlling peripheral vascular tone In human skin, CGRP induces slowly developing local reddening which may last for hours (see Brain et al, 1985). Brain et al, 1985 reported that intradermal injections of CGRP in humans at femtomole doses increased blood flow (flare reaction) which were very prolonged and with potencies similar to that of prostaglandin-E2. Weidner et al, 2000 also demonstrated that CGRP, administered by microdialysis, produced dose-dependent increases in vasodilatation. The long-lasting and widespread vascular effects of CGRP may reflect gradual diffusion of the peptide which may exert its actions on both the endothelium and vascular smooth muscles. It is anticipated that CGRP will play crucial roles in inflammation during the late stages whereas SP, most likely, will act more immediately during inflammation. The induration which may occur with the administration of CGRP could be ascribed to accumulation of leukocytes (Wallengren and Hakanson, 1987). CGRP has been found stable in interstitial tissue fluid (Brain and Cambridge, 1996) which could explain its long-lasting effects. Both Weidner and Wallengren agree that CGRP does not have sensory effects (pain, itch producing), and is not mediated by histamine.

As the concentration here is on the neuropeptide, CGRP as the principle therapeutic tool in the treatment of wounds, the following statements will be directed towards this goal.

6. The Biological Effects of CGRP.

One of the most prominent effects of CGRP in vivo is its vasodilator actions on small and large vessels. Intradermal injection causes a long-lasting erythema even in femtomol-to-picomol doses that could be inhibited by the antagonist CGRP 8-37 indicating a direct effect on the CGRP receptors that are present on arteriolar smooth muscle and endothelial cells. The regulatory properties of CGRP on the dermal microvascular homeostasis seem to be at least in part mediated by induction of nitric oxide (NO; Khalil and Helme, 1992; Bull et al, 1996). This suggests that NO may provide an additional link between the neuronal system and the skin. Alternatively, it has been proposed that NO may be necessary for either the release of CGRP from sensory nerves or its subsequent pathway. CGRP enhances endothelial cell proliferation and may therefore participate in angiogenesis during physiological and pathophysiological events such as ischemia, wound healing and inflammation. CGRP exhibits modulator activities in immune cell functions. Phagocytosis by cultured peritoneal mouse macrophages is enhanced by CGRP. CGRP stimulates adhesion of human neutrophils and monocytes. It also is thought to potentiate accumulation of neutrophils and edema formation induced by proinflammatory mediators although some human research disagrees with this (see Lundeberg, 1993; Weidner et al, 2002). Finally, the availability of neuropeptides to trigger cellular responses is effectively controlled by neuropeptide-specific peptidases.

In both animals and humans groups of investigators at the Karolinska Institute in Stockholm, Sweden (the Stockholm Groups) have shown the potent effects of CGRP on healing of ischemic flaps and wounds. Electrical stimulation with/without CGRP has also been shown to be efficacious in wound healing. It has been demonstrated that electrical stimulation causes release of neuropeptides, particularly CGRP. Early work in the 1980s revealed that:
1. In rats, flaps fail to heal when sensory nerves are desensitized by pre-treatment with CAP (see “Study Section”; Kjartannson et al, 1987), suggesting the role of neuropeptides in delayed wound healing
2. Transcutaneous electrical nerve stimulation (TENS, using 80 Hz and high intensity stimulation) increases blood flow and flap survival in rats (Kjartannson et al, 1988a)
3. Both CGRP and TENS increased cutaneous blood flow in a musculocutaneous flap of a rat with CGRP appearing more potent (Kjartannson et al, 1988b)
4. CGRP induces arterial dilatation and increased skin flap survival in the pig (Heden et al, 1989)
5. TENS stimulated healing of ischemic skin flaps in humans, demonstrating a potent vasodilator and anti-necrosis effect using a 80 Hz and moderate (“tingling sensation”) intensity stimulation (Lundeberg et al, 1988).

According to Raud et al (1991) one of the most important mechanisms regarding the action of CGRP is its anti-inflammatory properties. CGRP was found to inhibit edema-promoting action of inflammatory mediators, e.g., histamine, leukotrine, in vivo in the hamster cheek pouch, rat paw and human skin. The effect of CGRP was present in nanomolar dose range, and was mimicked by activation of sensory nerves (C-fibers) by acute capsaicin (CAP) which caused release of endogenous CGRP-like immunoreactivity (IR). This anti-inflammatory response contrasts with the pro-inflammatory reaction of CGRP when it is released from sensory nerves by antidromic stimulation of the nerves or by acute CAP. In this regard, CGRP participates in “neurogenic vasodilatation”, also an essential mechanism in wound repair (see Richards et al, 1997; Khalil and Helme, 1996). Thus, both pro-inflammatory and anti-inflammatory reactions to exogenous CGRP (intradermal) and endogenous CGRP (released by acute CAP; Holzer, 1998) play a role in wound healing.

Another series of papers in the 1990s again demonstrated the efficacy of CGRP in treating ischemia and inducing survival of flaps in rats (Gherardini et al, 1995, 1996, 1998a). These authors emphasized that CGRP caused was enhanced in the presence of ischemia. The latter suggests that there is post-terminal up-regulation of CGRP receptors in the presence of ischemia and that CGRP is involved in the adaptive response to ischemia. Jernbeck and Dalsgaard (1993) administered CGRP intravenously to patients with surgical or traumatic flaps. The patients showed compromised circulation due to ischemia and/or venous stasis. The treatment with CGRP (also TENS) improved tissue circulation as observed with laser Doppler flowmetry (LDF) but CGRP was more potent. It was interesting to note that CGRP infusions resulted in increased blood flow in the flap but not in corresponding control tissue. This suggests that there is altered sensitivity to CGRP in tissues with compromised circulation. Higher sensitivity to low dose CGRP can be found under conditions of hypoinnervation and denervation of the microvessels. The release of CGRP from sensory nerves is increased during ischemia and this has been suggested to enhance tissue repair, the adaptive response. Conversely, this adaptation is reduced after CGRP is depleted by chronic exposure to CAP (see “Study Section”).

According to these authors, CGRP acts as a local factor stimulating endothelial cell proliferation and is important in the formation of new vessels (angiogenesis). CGRP also increases the blood flux in ischemic tissue, promoting the survival of experimental and clinical flaps. The importance of CGRP in these conditions is further supported by the observations of Raud et al, 1991 who observed that the beneficial effect on flap survival and anti-inflammatory responses were blocked by CGRP-receptor antagonists. CGRP is effective in inhibiting histamine release from mast cells. Mast cells express CGRP receptors and release various cytokines and chemotactic factors. It is possible that the CGRP-induced decrease of flap neutrophil accumulation is dependent on the inhibition of the release of mast cell chemoattractants. In addition to “stabilization of mast cells”, CGRP inhibits the entirely leukocyte-dependent plasma extravasation evoked by the specific leukocyte chemoattractant leukotriene B4 (Raud et al, 1991). Moreover, it is postulated that CGRP may suppress the expression of leukocyte adhesion molecules such as endothelial selectins and/or leukocyte integrins. Certainly, promotion of endothelial cell growth and inhibition of cell types such as lymphocytes, macrophages and epidermal Langerhan cells will contribute to healing.

Engin (1998; not from the Stockholm group) showed that CGRP, administered intradermally around wounds in rats, had a trophic effect on healing by increasing the contraction rate. Engin ascribed improved healing of wounds by CGRP as a form of local action on sensory terminals near the defect to produce its trophic action rather than to the hyperesponsiveness of exogenous CGRP on sensitive ischemic tissue. Moreover, CGRP can have a direct action on endothelial cells, thus releasing NO, and on vascular smooth muscle, both increasing its trophic and immunomodulatory functions. The question whether nerve terminals in the vicinity of the microvessels must be available for CGRP to be effective is not supported by the work of Gherardini et al, 1998b; Ardron et al, 1991-see below) and by the fact that CGRP is effective in the presence of C-fiber degeneration as observed after pre-treatment with CAP, and under conditions of aging, diabetes and other conditions with small fiber neuropathy (see “Study Section”).

7. Venous Stasis and Diabetic Ulcers in Humans.

New advances in the treatment of two of the most common forms of skin wounds, namely chronic venous stasis and diabetic ulcers have been described. Both conditions are related to impairment of small sensory fibers, i.e., C-fibers (Ardron et al, 1991; Parkhouse and LeQuesne, 1988) and, hence, to reduction of neuropeptide control of the microcirculation and associated tissues.

8. Venous Stasis Ulcers.

Chronic venous insufficiency of the lower limbs may produce ischemia and ulceration that respond poorly to medical and surgical intervention, leaving the physician with limited alternatives other than chronic, and costly, wound care. Alternative treatments have emerged which use low level TENS which have been effective in promoting healing of diabetic ulcers and ischemic flaps (see above) and absorption of peptides through the skin (Costello and Jeske, 1995). More recent studies have shown that two peptides, CGRP and Vasoactive Intestinal Polypeptide (VIP), released by C-fibers, increased perfusion of ischemic tissue and survival of flaps with compromised circulation (see Gherardini et al, 1998b). Interestingly, Gherardini and his colleagues administered CGRP and VIP transdermally by IONTOPHORESIS. The mechanism of iontophoresis is based upon the migration of electrically charged particles across biological membranes to follow the flow of a direct electric current. Originally described to transport various ions using relative inert buffers (e.g., sodium nitroprusside driven by negative current with methylcellulose as the buffer to transport the NO donor-personal observations), iontophoresis is now used to administer various molecules and peptides (Cross and Roberts, 1995). In Gherardini's studies, the iontophoresis electrodes were 40 cm² and contained a drug reservoir of 3 ml mixture of-1 ml CGRP (3×10⁻⁹ M) and 1 ml of VIP (3×10^{-5s} M) added to the reservoir of the positive electrode and sodium phosphate buffer (3 ml) was added to the reservoir of the negative electrode. The current was delivered at a frequency of 3000 Hz for a period of 1 ms which was followed by a rest period of 1 ms. The polarity of the treatment electrode was changed after each treatment. The intensity was sufficient to produce paresthesias, thus sufficient to elicit vasodilatation by itself. The authors of the paper set forth in “Study Section” have demonstrated that the cathodal current of iontophoretic chamber containing an inert substance can produce substantial vasodilatation with low current intensities delivered over prolonged periods of time and that this vasodilatation can be attributed to the failure of release of neuropeptides such as CGRP when the skin is pre-treated with CAP.

9. Diabetic Ulcers.

Electrical stimulation of the skin (TENS) improves healing of diabetic ulcers. In humans, Lundeberg and his colleagues (1988, 1992, 1993) demonstrated that constant current with a frequency of 80 Hz, pulse width of 0.4-1 ms, and an intensity below pain threshold promoted vasodilatation and wound repair. Among the diabetic ulcer patients, the current was applied 20 min twice daily just outside the ulcer area. Blood flow was always recorded by LDF (see above and “Study Section”). Lundeberg (1993) claimed that successful healing of diabetic ulcers can occur within 12 weeks of treatment and need not exceed 40 min/every day (see also Kloth and Feeder, 1988) although this length of time is in some dispute, Accepting the possibility that the time of treatment is too short, Lundeberg suggests a longer treatment time may result in more significant enhancement of healing but that it seemed unlikely that it need to exceed 40-60 min, 5-7 days/week. Their conclusion (Lundeberg (1993)) was: “the results implicate that sensory stimulation influences, through the release of vasodilator peptides (particularly CGRP), the proliferative and functional capacity of fibroblasts, leading to increased collagen synthesis, and increased receptors of TGF-β, thus promoting the healing of wounds”. Both Lundeberg and Gherardini claim that electrical stimulation can enhance protein synthesis as well as influence fibroblasts, and migration of epithelial cells from the edge of the wound, thereby augmenting wound healing. Others have also reported that TENS of low intensity nature could also accelerate healing of ulcers of various etiologies (e.g., Carley and Wainapel, 1985; Stromberg, 1988).

10. Ulcers Due to Aging.

Pressure ulcers are a serious and all too common complication of immobility among the elderly. In the late 1980s, the prevalence of pressure ulcers was 3-11% in acute care hospitals and nursing homes (Allman, 1989). In this study, as many as 50% of patients with pressure ulcers were over the age of 70. Among geriatric patients and nursing home residents, pressure ulcers were associated with a four-fold increase in the risk of death. The in-hospital mortality rate among patients with pressure ulcers was between 23 and 37%. Later, Brandeis et al, 1990 evaluated 19,889 elderly residents of 51 nursing homes. Among all residents admitted to nursing homes, 11.3% possessed a stage II through stage IV pressure ulcer. In those admitted without a pressure ulcer, the 1-year incidence was 13.2%. This increased to 21.6% at the end of 2-years. Many healed within one year but others required re-hospitalization and were at a high mortality risk.

Many risk factors have been identified which include immobilization, malnutrition, incontinence, age-related changes in skin such as loss of dermal vessels, thinning of the epidermis, etc. Four factors have been implicated in the pathogenesis of skin breakdown: pressure, shearing forces, friction and moisture.

One significant risk factor has been left out of the equation regarding pressure ulcers and impaired wound healing which are common in the elderly (Eaglstein, 1989). That is, the role of the microcirculation in association with its innervation, namely small primary afferent neurons (i.e., C-fibers). Aged humans reveal robust impairment of C-fibers and hence of neurgenic vasodilatation, resulting in failure to appropriate perfuse cutaneous tissue (Helme and McKernan, 1985; Ardron and Helme, 1990). Ardron et al, 1991 showed impairment of microvascular responses in elderly people with varicose leg ulcers. They claim that the nociceptive system of primary unmyelinated nerves probably prevents damage from repetitive minor injuries which go otherwise unnoticed (a most likely relationship to diabetic ulcers). This nociceptive system is responsible for vasodilatation of the microvessels and has a “trophic” role mediated by the neuropeptides that are released by the C-fibers; the latter functions by acting as growth factors for epidermal cells and fibroblasts. The failure of this same system is found among diabetic patients with leg ulcers (Parkhouse and LeQuesne, 1988; Walmsley et al, 1989). Ardron et al, 1991 concluded that manipulation of the nociceptive C-fiber system by topical treatment with neuropeptides, may speed-up the healing of chronic skin ulcers. This proposal was certainly supported by the work of Gherardini et al (1998b) who showed that iontophoresis of CGRP and VIP, two neuropeptides released from C-neurons, promote vasodilatation, trophic influences and immunomodulation in the region of the ulcers, and improved healing of venous stasis ulcers.

In animals, there has been substantial support for the premise that the capacity to maintain appropriate inflammatory and repair processes in skin ulcers is decreased with age. This work has principally been conducted by Khalil and her colleagues in Melbourne. These investigators claim that neurogenic inflammation (in humans, more specifically “neurogenic vasodilatation”) is essentially a protective response in the process of tissue repair. A major part of a normal neurogenic inflammation response in tissue is dependent on intact unmyelinated primary afferent innervation (see Kjartannson et al, 1987). One of the most interesting aspects of Khalil's work is the observation of post-terminal changes following impairment of C-fibers in the presence of induced skin ulcers in aged rats. Thus, up-regulation of receptors of the neuropeptides may be responsible for the observed more robust vasodilator effect of CGRP and SP when they were perfused in the wound, an effect observed in capsaicin pre-treated and aged animals (Khalil et al, 1994; Khalil and Helme, 1996; see “Study Section”). Further, the increased responsiveness may be ascribed to changes in endothelial mechanisms involving nitric oxide (NO). Thus sensory nerves mediating CGRP and SP, and NO are involved in the neurogenic inflammatory responses (Khalil and Helme, 1992; Bull et al, 1996). Bull et al showed that endothelial NO Synthase is expressed in the microvascular endothelium of normal human skin and that dermal microvascular endothelial cells release NO constitutively and in response to vasodilator neuropeptides.

It is proposed that pre-terminal changes in nociceptors (C-fibers) also occur and are most likely due to degeneration of epidermal fibers which are predominantly capsaicin-sensitive C-fibers and may not be due to altered axonal transport from the DRGs as contemplated by Khalil and her colleagues. Functional impairment of C-fibers and reduced Epidermal nerve density are noted among subjects with Raqynaud's Phenomenon, diabetes, obesity, aging and spinal cord injury, all of whom are at high risk for wound occurrence and delayed healing of wounds and flaps (see “Study Section”).

As observed in animals and humans with ischemic flaps, venous stasis and diabetic ulcers, TENS treatment, using low frequency (e.g., 8 Hz) and high intensity current accelerated wound healing in aged rats and rats pre-treated by CAP by peripheral activation of sensory nerves (Khalil and Mehri, 1996). Please note the differences in TENS use between the many investigators including ourselves (see “Study Section”).

11. Nerve Growth Factor (NGF) & Nitric Oxide (NO) in Wound Healing.

Nerve growth factor is instrumental in the synthesis of sensory neuropeptides in the DRG. Li et al, 1980 report that removal of the submandibular glands of mice retards the rate of wound contraction and that communal licking of wounds accelerates wound contraction in intact animals. It is suggested that the submandibular gland NGF (a very high molecular weight substance) is secreted in saliva. NGF is made by other non-neural tissue such as fibroblasts which are a prominent feature of granulation tissue which also seems to play some biological role in the process of wound healing. Li applied 50 ul of a solution (110 ug/ml) of the protein in a phosphate buffer topically to wound areas which produced markedly accelerated healing. Lawman et al (1985) state that NGF accelerates the early cellular events associated with wound healing. They comment that the outcome of Li's work could be due to a direct or indirect generation of chemotactic factors that acted upon the cellular components involved in wound healing. In fact, these authors suggested that ANY chemotactic factor might mediate this sequential accumulation of cells and would in turn promote wound healing (see chemotaxis above).

As has been described above, iontophoresis for transdermal administration of sodium nitroprusside, a NO donor, produces endothelium-independent vasodilatation of the microcirculation in normal human subjects. Unfortunately, the degree of vasodilatation produced is no more potent than the vasodilatation created by electric current with only an inert substance in the reservoir (‘the control”), suggesting that C-fiber activation which releases CGRP and causes endothelium-dependent (NO-induced)- and -independent (vascular smooth muscle-induced; Bull et al, 2000) vasodilatation is just as effective as NO alone. However, in the presence of C-fiber impairment (e.g., diabetes) when electrical stimulation (the control) can fail to produce vasodilatation, iontophoresis of NO induces a marked vasodilatation which may be used to effectively dilate the microvessels and assist in the healing process. In fact, in ultra-violet induced inflammation, NO, derived from the C-fibers and also expressed by the endothelium, and CGRP contribute substantially to the invoked neurogenic vasodilatation and healing (Benrath et al, 1995).

The amino acid L-Arginine (LA), a unique substrate for NO synthesis, has biological effects including secretagogue actions on several endocrine glands, enhancement of cellular immune system and anti-catabolic activities following stress and trauma (Shi et al, 2003). In addition, supplemental LA improves healing in both animal and humans wounds; it is directly involved in many regulatory mechanisms relevant to wound healing such as angiogenesis, cell proliferation and collagen metabolism. The role of NO in the healing process is underscored by studies showing that healing is impaired during systemic administration of a NO Synthase inhibitor (Schaffer et al, 1996) and in inducible NOS knockout mice (Yamasaki et al, 1998). In diabetes, impaired wound healing may be related to high blood sugar which hinders cell proliferation and collagen formation, decreased expression of multiple growth factors, impaired phagocytosis leading to increasing rates of infection and local wound apoptosis. Furthermore, wound NO synthesis is impaired and may contribute to observed failures to heal. Shi et al showed that LA supplementation can improve wound healing of diabetic rats. There was a statistically marked effect on wound strength, wound collagen expression and expression of Type I-III procollagen mRNA. It is possible that NO directly regulates expression and activity of several transcription factors including NF-kβ, which in turn modulate synthesis and release of multiple-wound healing-participating cytokines/proteins. Wound levels of NOx and protein are lower in diabetic wounds. LA administration increased NOx levels to normal values which is accompanied by enhanced wound NO synthesis.

Luk et al (2005) propose that inducible NOS (iNOS) expression is noted in faster healing chronic leg ulcers. They claim that NO exerts beneficial effects on many processes of healing including bactericidal effects, angiogenesis, epithelialisation and ECM formation. A seminal paper related to NO and wound-healing was presented by Isenberg et al, 2005. Some of the salient points are listed:
1. Wound contraction is a major contributor to closure of the wounds. In excisional wounds, closure is delayed by iNOS inhibition.
2. Wound fibroblasts, which are responsible for collagen production and deposition in wounds, show increased NO production, suggesting an association between NO and the process of collagen production.
3. NO, using the NO-donor sodium nitroprusside, increased production of cGMP indicating that NO and cGMP promote growth factor-induced DNA synthesis in dermal fibroblasts and, hence, presents another mechanism by which NO may promote wound-healing.
4. In a model of an injured Achilles tendon treated with NO and flurbiprofen, the NO-treated group showed better organization of extra-cellular collagenous matrix in the tendon samples than in the vehicle group. Others have reported that topical NO, delivered as glycerol trinitrate, decreases pain and increases range of motion in tendonitis.
5. Isenberg et al claim that the lack of studies investigating the application of NO to cutaneous wounds is due to a lack of vehicles by which a NO-donor can be delivered. Aged rat wounds treated with topical NO showed delayed healing compared to saline-treated controls. Conversely, diabetic mice wounds treated with topical NO did not differ in length of time of healing compared to controls, though the NO-treated wounds showed increased granulation-tissue and collagen deposition. (It is obvious that iontophoresis of NO, which can provide longer term treatment including TENS, was not a treatment choice)
6. That NO may have a beneficial role in wound healing can be inferred from studies in which ischemic tissue loss was treated successfully with NO. Direct administration of various NO-donors appears to extend tissue survival after ischemic insult.
7. Additional support for the role of NO in flap survival, tissue preservation, and wound-healing is provided by studies of the NOS inhibitor, L-NAME, which resulted in marked decreases in vessel diameter and blood flow in skin flaps of animals; under pathologic conditions of NO deficiency as occur in diabetes or malnutrition, an association with delayed healing and impaired extracellular matrix deposition is documented.
8. Substrate availability as described by Shi et al (see above) with respect to L-Arginine which increases wound strength among diabetic animals may overcome some rate-limiting NO production problems noted in diabetic foot wounds.
9. Dermal injection of histamine (a mediator of a vasodilator inflammatory responses) led to increased local NO and cGMP levels as measured by microdialysis. Isenberg claims, that as with other modulators of vascular cell responses, the particular cell(s) response to NO, and, thus its effects on a particular wound, will depend on the context.

An additional area of interest in CGRP is centered about the fact that human CGRP is a very potent microvascular dilator of the skin in both animals and man (Brain et al, 1985). CGRP is released from small sensory nerve fibers (e.g., C-fibers), most of which are capsaicin-sensitive. Thus, capsaicin-sensitive stimulation by such modalities as heat, capsaicin, and electrical stimulation induces release of CGRP and subsequently robust neurogenic vasodilatation, the central focus of our clinical intentions in humans.

A number of disorders associated with small sensory nerve neuropathy (e.g., pre-diabetes, diabetes, obesity without hyperglycemia, Raynaud's Phenomenon) reveal markedly reduced CGRP-immunoreactive nerve fibers in the skin and, hence, reduced neurogenic vasodilitation. This abnormality is a fundamental factor in skin ulcer production, impaired wound healing, failure of skin flaps to heal, etc. A basic thesis is that replacement therapy with CGRP will be an effective treatment regimen for these disorders.

Brain and Grant (2003) suggest that replenishment of CGRP is limited by methods of administration (e.g., i.d., microdialysis). In addition to the methods of administration described above, other injection free methodologies utilizing passive (e.g.; a polymer which can be used on the surface of the skin) or active (e.g.; iontophoresis as shown in FIGS. 2-3) transdermal administration of CGRP for the treatment/prevention of neuropathic ulcers (e.g.; from C-fiber neuropathy and the sequelae of severe microvascular disease as is often observed in Raynaud's Phenomenon).

Passive transdermal systems are employed to diffuse drug through the skin where it can act locally or penetrate the capillaries for systemic effect. This technology is only effective with small molecule drugs which can be utilized with skin patches containing such substances as nicotine, clonidine, and nitric oxide (NO) donor patches. However, lotions, ointments and sprays may also be useful.

In active transdermal systems, drug molecules of significant molecular weight are forced through the skin. By using an applied force (e.g., ultrasound or electrical stimulation-iontophoresis already described), active transdermal systems are capable of delivering proteins and other large molecules through the skin, and, if necessary, into the blood stream.

The usual doses used for i.d. administration of CGRP in human skin are considerably lower than doses which would produce systemic effects such as raised heart rate, lowering of blood pressure. This would also utilize similar lower doses with transdermal administration, that is, doses eliciting a strong microcirculatory vasodilatation without systemic actions.

By way of example, and not by way of limitation, relative to a human hand, a treatment contemplated for Raynaud's disease and phenomenon is illustrated in FIG. 3.

Study Section

A study, titled “: The Prevalence of Somatic Small Fiber Neuropathy in Obesity”, will be briefly summarized and then more expansively discussed.

Background: Somatic cutaneous small sensory fiber neuropathy (SSFN) can be an early manifestation of Impaired Glucose Tolerance and Diabetes Mellitus and/or insulin resistance among obese subjects and is often associated with pain, wound occurrence, and impaired wound healing. It is yet unclear as to whether SSFN is prevalent among obese individuals without glucose and/or insulin dysregulation despite abundant evidence of delayed wound healing.

Objective: To observe whether there is hypofunctioning of stimulated capsaicin-sensitive cutaneous nerves (small sensory fibers) in obese subjects with/without hyperglycemia and hyperinsulinemia.

Design, Setting and Participants: Fifty eight morbidly obese and 15 lean subjects were recruited for small fiber testing of the forearm in a cross-sectional study. Hyperglycemia was observed in 35 obese subjects. Of 25 obese subjects, hyperinsulinemia was noted in 15, 14 of which were hyperglycemic. No subjects demonstrated symptoms/signs of neuropathy over the hairy skin of the forearm. In fact, a neurological examination revealed that 37 subjects were asymptomatic in the legs and only 4 complained of a neuropathic pain in the foot. Virtually all subjects were exposed to a set of capsaicin-sensitive tests and measures which were identified by capsaicin desensitization procedures. These tests, conducted while in a supine position in bed, examined the two principle roles of curaneous SSFs, namely conveying pain signals to the CNS and controlling local neurogenic vasodilatation (flare; axon-reflex).

Main Outcome Measures: Heat-induced pain was assessed by verbal reports of sensation after accommodation and heat-, capsaicin-, and transcutneous stimulation-induced blood flow was measured by laser Doppler flowmetry with probes placed at the site of stimulation and 1 cm remote from the site, the latter to evaluate flare latency and intensity of flare.

Results: Significant depression of pain and flare responses were observed in the obese subjects in all but one test. Decreased pain and flare responses were noted in all subjects without hyperglycemia and hyperinsulinemia. Age negatively correlated with capsaicin-induced flare in both the obese and normal groups.

Conclusion: SSFN was prevalent in the cohort of morbidly obese subjects in a skin area without neurological symptoms or signs and in subjects with/without hyperglycemia and hyperinsulinemia. SSFN may be a serious factor in observations of impaired wound healing among obese subjects, a particularly worrisome problem in an obese aging population given the propensity for small fiber impairment in aging subjects. Small fiber impairment in the younger obese population may signal an early aging phenomenon.

Introduction to the Study

The past quarter century has witnessed a dramatic increase, now reaching epidemic proportions, in the prevalence of a cluster of inter-related metabolic disease states that are associated with obesity, e.g., insulin resistance, impaired glucose tolerance, Type II Diabetes, and hyperlipidemia. Obesity related disorders kill some 300,000 persons each year. This trend is worsening, particularly in North America where the prevalence of overweight adults has increased by more than 50% in the past decade. Without question, chronic diseases associated with obesity and diabetes represent a heavy and growing burden to society in terms of both direct medical costs and significant morbidity and mortality rates.

Central to the disease states associated with glucose/insulin dysregulation is the role of the small diameter cutaneous primary afferent neurons, comprised of both myelinated (Aδ) and unmyelinated (C) nociceptors. Though they are the largest group of primary afferent neurons in human cutaneous nerves, they can not be assessed by the quantitative measures routinely used to evaluate large fiber neuropathy such as vibratory threshold, nerve conduction and sensory latency tests.

Descriptions of small fiber neuropathy usually implicate impairment of both somatic and sympathetic nerves. However, this paper limits its focus to somatic small fiber neuropathy (SSFN), specifically cutaneous nociceptors. SSFN is well known as a common disorder in both pre-diabetes, i.e., impaired glucose tolerance (IGT)5 and in fully developed diabetes. Some claim that it is the small nerve fibers that are notably impaired in IGT, whereas both small and large fibers are affected in diabetes. Indeed, small nerve fibers may offer the earliest detectable sign of impending neuropathy in persons with glucose dysmetabolism and/or with insulin resistance. The involvement of small fibers before large fibers is confirmed in nerve biopsy studies which found a predominant loss of Aδ and C fibers earlier than the loss of large myelinated (Aα, Aβ) fibers.

It has been observed that the most frequent abnormality among asymptomatic and symptomatic (painful neuropathy) diabetic subjects was an elevated threshold for thermal sensation in the foot, a sign of small fiber dysfunction. It must be emphasized that although small fiber neuropathy was often associated with neuropathic symptoms like paresthesias and pain, many people were asymptomatic. Others have found that some forms of SSFN do not necessarily follow a “dying-back process” with length-dependency (distal to proximal) progression with duration of diabetes; they may be diffuse, multifocal, or generalized.

The Aδ- and, more so, C-fiber primary afferent neurons involved in SSFN are notably nociceptors responding to noxious mechanical, thermal, and irritant chemical stimuli; but they can also be excited by non-noxious stimuli such as heat and transcutaneous electrical stimulation. Although C-fibers convey pain signals to the CNS, they also have an “efferent, or effector” function. Under normal physiological conditions, C-fibers control vasodilatation of the skin microvasculature (arterioles), mediating axon-reflexes locally and/or centrally (dorsal root reflexes) by release of neuropeptides (e.g., calcitonin gene-related peptide, CGRP) from the terminals of the nociceptive afferents. As a consequence, microcirculatory impairment at least in the upper extremity is related to the dysfunction of small fiber neuron, without coexisting microangiopathy. This exists in obese subjects with hyperglycemia, hyperinsulinemia and insulin resistance without hyperglycemia, as well as in obese subjects without insulin and glucose dysregulation.

To test the function of small somatic (not sympathetic) nerve fibers, capsaicin, a naturally occurring pungent ingredient of chili peppers, has been applied acutely to the skin. Corresponding to the concentration, topical capsaicin causes spontaneous burning pain, sensitization, and a hyperemia or “flare” reaction; later, following chronic administration, nociceptor desensitization occurs. Most likely, this is due to the human epidermis containing a high density of capsaicin (vanilloid or TRPV1) receptors and axons labeled with the pan-neuronal marker, protein gene product (PGP 9.5). This suggests that the epidermis is rich in capsaicin-sensitive nerves.

In skin, these capsaicin-sensitive nociceptive afferents play a significant role in sensory function, particularly a nocifensor role in tissue protection due to their ability to: 1) convey pain signals to the CNS upon stimulation by noxious mechanical, thermal, and irritant chemical stimuli; 2) elicit a pruritic reaction; 3) cause sensitization of the skin (e.g., to heat) decreasing the pain threshold, a process defined as primary hyperalgesia; 4) evoke an axon-reflex flare or neurogenic vasodilatation. All four of these reactions, acute heat pain, pruritis, sensitization, and flare reactions, are C-fiber mediated, apparently by utilizing different sub-sets of C-fibers.

Specific noxious and non-noxious stimuli are applied to the skin to activate nociceptive afferent neurons to induce a spreading hyperemia, which is visible as a neurogenic vasodilatory, or flare, response. Assessment of the axon-reflex flare by noxious heat- and capsaicin-stimulation and by non-noxious transcutaneous electrical stimulation can be used to measure C-fiber function. While objective measurement of the flare reaction can be accomplished by visual assessment, introduction of laser Doppler flowmetry (LDF) has made it possible to monitor the flare response continuously in a small area of the skin, thereby facilitating the study of the axon-reflex in terms of flux.

Continual exposure to topical capsaicin leads to desensitization of nociceptive afferent neurons and, thus, increases the heat pain threshold and virtually abolishes the neurogenic vasodilatory response to noxious mechanical, thermal or irritant chemical stimulation as well as to non-noxious stimulation. Such outcomes provide evidence that these tests can be used to assess the function of C-fibers and SSFNs. Capsaicin-induced desensitization is ascribed to morphological damage of Epidermal Nerve Fibers (ENFs) which contain a high concentration of nociceptive afferent neurons.

Some morbidly obese subjects with or without hyperglycemia and/or hyperinsulinemia show a profound SSFN in capsaicin-sensitive tests of pain and flare. Functional impairment of small fibers can be observed in both the forearm and thigh to the same degree, suggesting that the SSFN was a generalized phenomenon. This study presents results in the following sections to support the view that obese subjects with or without hyperglycemia and/or hyperinsulinemia demonstrate SSFN.

Methodology

Subjects: Fifteen (15) normal and fifty-eight (58) morbidly obese subjects were recruited. The morbidly obese subjects had a BMI>35 and a waist/hip ratio consistent with visceral obesity. Table 1 lists the population characteristics.

TABLE 1
Population Characteristics

| Characteristics | Normal Subjects n = 15 | | Obese Subjects n = 58 | |
|---|---|---|---|---|
| | Mean | Range | Mean | Range |
| Age (years) | 32.40 | 20.00–57.00 | 45.30 | 29.00–68.00 |
| Males BMI | 22.80 | 20.00–25.00 | 48.50 | 36.00–68.00 |
| (kg/m²) | | | | |
| Females BMI | 21.30 | 19.00–25.00 | 48.60 | 35.00–65.00 |
| (kg/m²) | | | | |
| Males Waist/Hip | 0.89 | 0.83–0.92 | 1.00 | 0.89–1.10 |
| Ratio | | | | |
| Females Waist/Hip | 0.76 | 0.72–0.79 | 0.83 | 0.76–1.10 |
| Ratio | | | | |
| Glucose (mg/dl) | 85.00 | 81.00–88.00 | 137.00 | 68.00–339.00 |
| HbA1C | | | 6.90 | 4.80–11.10 |
| Cholesterol | 174.00 | 101.00–256.00 | 197.00 | 130.00–270.00 |
| Triglyceride | 74.00 | 43.00–120.00 | 229.00 | 45.00–505.00 |
| | Number | Percent | Number | Percent |
| Hyperglycemia | | | | |
| Diabetic | 0 | 0% | 21 | 36% |
| IGT | 0 | 0% | 14 | 24% |
| Hyperlipidemia | 6 | 40% | 38 | 66% |
| Sleep Apnea | 0 | 0% | 15 | 26% |
| Tobacco Use | 0 | 0% | 14 | 24% |

The normal subjects were excluded if they exhibited hyperglycemia, hyperinsulinemia, sleep apnea or tobacco use, whereas obese subjects were accepted with various conditions related to the metabolic syndrome. Peripheral vascular disease or an ankle-brachial index of <0.9 were cause to exclude any subject. All qualified subjects received a clinical neurological examination of the peripheral nervous system of the upper and lower extremities.

Cutaneous Blood Flow Measurement: Cutaneous blood flow can be measured using a DRT 4 Laser Doppler Flowmeter system available from Moor Instruments of Devon, England. A fiber optic probe directed laser-generated light of a wavelength of 780 nm-820 nm to the skin surface. As the light reflected from moving blood cells, it underwent a shift in frequency (Doppler Effect) that is dependent on the number and velocity of the moving blood cells. Blood flow was measured in arbitrary units (AU). The probe was placed into a recess in the center of a stimulating chamber (e.g., perspex, thermode) to measure “direct flow” and in a recess 10 mm outside the edge of the stimulator to measure “indirect flow” or the axon-reflex. This probe configuration was used for all tests. Direct and indirect flow was measured under three modes of stimulation: heat via a contact thermode, topical capsaicin application via a passive iontophoretic chamber, and transcutaneous electrical stimulation via an active iontophoretic chamber.

Protocol: Experiments were conducted in a 24-26° C. room with the subject reclined in a hospital bed with one or two pillows under the head. Right and left forearms were tested alternately with the stimuli being placed aside any prior hyperemic skin. The hairy skin of the proximo-lateral forearm was continually cleansed with an alcohol swab. Prior to each test, 60 seconds of baseline temperature and blood flow were recorded. These values were subtracted from peak blood flow values to yield a “net” flux, the value reported in the Results Section.

Heat Stimulation: Before testing, each subject underwent a training trial to ensure their prompt verbal report of the various sensations accompanying rising temperature (e.g., warm, itch, hot, burn). Heat was delivered by a circular brass contact thermode (10 mm diameter) centered in a 35 mm stabilizing collar placed on the forearm. A probe was placed in the center of the thermode and a second probe was placed 1 cm from the thermode. Two forms of stimulation were used: heat-ramp and heat-step.

In the heat-ramp procedure, the thermode temperature was fixed at 32° C. for 1 minute followed by a 25 second ramp to 44° C. that was then maintained for 20 minutes. 38 The subject reported continuously on perceived sensations. The time of each report and the maximum net hyperemic response as determined by LDF were recorded.

In the heat-step procedure, basal blood flow was monitored at resting skin temperature and later at 32° C. The temperature was raised in increments of 2° C. per minute until a maximum temperature value of 48° C. was reached. Sensory responses (specifically, heat pain threshold, HPT) and peak net flows at each increment were documented.

A second heat-step test followed an acute application of topical capsaicin to the skin to measure sensitization by HPT, i.e., primary hyperalgesia. Two normal and obese subjects complained that the pungency from the capsaicin was extreme and, as a consequence, the heat-step test for hyperalgesia was not conducted.

Topical Capsaicin: Capsaicin, 8-methyl-N-vanillyl-6-nonenamide, is a vanilloid neurotoxin that targets somatic small primary afferent neurons. Capsaicin (1% in 75% ethanol, 25% saline) was applied topically for 45 minutes through a central chamber (300 μl capacity, 1 cm diameter) in a clear circular plastic perspex adhered to the skin. Throughout the application, perceptual responses and peak flow values were obtained. The vehicle was applied at different skin sites to 6 normal subjects.

Transcutaneous Electrical Stimulation: A non-noxious, constant cathodal current was delivered by a platinum ring electrode in a central iontophoretic chamber. The central reservoir of the iontophoretic chamber was filled with Methylcellulose (2%), an inert vehicle used with other vasoactive compounds for iontophoretic drug delivery. An indifferent electrode was applied to the wrist. Progressive increases in current dose (millicoulombs, mC) were delivered using a current of 0.2 mA applied for 10, 20, 40, 80 and 160 sec. These represented doses of 2, 4, 8, 16, and 32 mC. A post-stimulus interval of 180 seconds followed each of the first four doses; a 240 second interval followed the highest dose. Peak flow at the end of each stimulus period was recorded.

Capsaicin-Induced Desensitization: Eight (8) normal subjects were recruited to ascertain the degree of capsaicin sensitivity of each of the tests described above. Each subject applied topical capsaicin to one forearm and thigh, and the vehicle to the opposing limb in 45 minute applications, 3-4 times daily for 5 days. The results of these experiments suggest that all tests revealed capsaicin sensitivity as presented, in-part, in FIG. 1.

Statistical Analysis: Group mean data for continuous variables, such as blood flow, temperature and latency were compared using unpaired t-tests (HPT, flare, latency) and one-way analysis of variance with Tukey's post-hoc multiple comparison tests for differences in dose response curves (electrical stimulation). Data for individual participants were standardized for each dependent variable using Z-scores, and Pearson's R2 correlation values with 95% confidence interval were used to detect relationships between variables. All statistical analyses were performed by means of SPSS statistical software (version 11.5.0). Statistical significance was determined by a two-tailed assessment. All p-values were designated within the figure (n.s.=p>0.05).

Results:

Demographics

Table 1 identifies the pertinent variables associated with age, sex, BMI, Waist/Hip Ratio, and levels of glycemia and lipidemia in the two population samples. Insulin values were obtained from 10 normals (all normal values) and 25 obese subjects. Of the 25 obese subjects, 15 were hyperinsulinemic, 14 of which were also hyperglycemic (IGT=9; DM=6). Hyperglycemia was noted in 35 obese subjects and none of the normal subjects. Of these 35 obese subjects, 14 were IGT and 21 were DM. Please note that 40% of the normal subjects revealed hyperlipidemia.

The neurological examination of 55 obese subjects revealed the following: 25 had no symptoms or signs of neuropathy; 11 reported no symptoms but demonstrated small fiber impairment; 1 reported no symptoms but showed large and small fiber impairment; two groups of 7 reported non-painful symptoms, usually in the foot, with either involvement of small fibers only or large and small fibers; 4 reported painful feet associated with small and large fiber neuropathy. There were no symptoms or signs of neuropathy over the area of skin tested.

Capsaicin-Induced Desensitization

Topical capsaicin was applied to normal subjects (n=8) 3-4 times daily for 5 days. When compared with the action of the vehicle alone, capsaicin-induced desensitization (Cap-D) to heat, acute capsaicin and transcutaneous electrical stimulation was pronounced for each stimulus based upon the following observations:
1. HPT increased to greater than the maximum temperature utilized (48° C.).
2. During the heat-ramp test, heat-induced flux decreased significantly under the thermode (direct flow) and also outside the thermode (indirect flow). (FIG. 1A).
3. An acute capsaicin challenge produced no pungency and virtually no increased blood flow (not illustrated).
4. At the indirect site, a non-noxious transcutaneous electrical (cathodal) stimulation utilizing the vehicle produced a rising dose (mC)-response (flux) curve. Following Cap-D the curve was flattened indicating an abolished neurogenic flare. (FIG. 1B)
5. Consequently, heat, capsaicin and electrical stimulation-induced responses of pain (pungency) and/or flare were considered capsaicin-sensitive neurogenic responses indicative of smallfiber impairment.

Acute Effects of Capsaicin
1. Quality of Sensation. Using criteria for capsaicin-induced pungency, normal subjects reported a 90% pungency level with approximately 80% perceiving burn/pain and 20% reporting a stinging/prickly sensation. Obese subjects reported only a 45% pungency level described as burn/pain in all but one subject. Six (6) subjects did not perceive any change in sensation (e.g., itch, warm). In normal subjects pungency and flare were not elicited by the vehicle alone.
2. Blood Flow (Flux). (FIG. 2A) Both direct and indirect flow sites showed a capsaicin-induced peak flow that was significantly lower in obese subjects compared to normals. The mean peak direct and indirect flow values for the normal and obese subjects are depicted in Table 2.

TABLE 2
Summary Data

| | Direct | | | | Indirect | | | |
|---|---|---|---|---|---|---|---|---|
| | Normal | | Obese | | Normal | | Obese | |
| Test | Mean | SEM | Mean | SEM | Mean | SEM | Mean | SEM |
| Capsaicin | | | | | | | | |
| Flux (AU) | 299 | 44.00 | 159 | 17 | 257 | 27 | 128 | 13 |
| Latency (min) | 11 | 2 | 21 | 2 | 12 | 1 | 26 | 2 |
| Heat Ramp | | | | | | | | |
| Flux (AU) | 313 | 50 | 226 | 19 | 155 | 32 | 59 | 8 |
| Heat Step HPT | | | | | | | | |
| Pre-Cap | 44.8° C. | 0.6 | 46.7° C. | 0.2 | | | | |
| Post-Cap - | 37.1° C. | 1.2 | 39.8° C. | 0.9 | | | | |
| Hyperalgesia | | | | | | | | |

3. Latency. (FIG. 2B) Both direct and indirect flow sites showed a capsaicin-induced vasodilatory response latency that was significantly delayed in obese subjects compared to normals. The mean latency for direct and indirect flows is described in Table 2. Thirty one (31) obese subjects demonstrated prolonged latencies (e.g., >20 min) to the flare response, i.e., greater than 2 standard deviations from the normal mean. Among these 31 subjects, 20 did not report pungency. Furthermore, 18 subjects revealed very low flow amplitudes, defined as a flux <100. Eleven (11) subjects demonstrated a flare latency at or near the maximum exposure time of 45 min or not at all. Of these 11 subjects, 8 reported no pungency. The normal subjects exposed to the vehicle did not demonstrate a delayed response. FIG. 2C depicts a significant (p<0.001) negative correlation between flux (indirect flow) and latency to the flare with a correlation coefficient of R²=0.401.
4. Capsaicin-Induced Hyperalgesia to Heat. Capsaicin caused a marked primary hyperalgesic response to heat in both the normal and obese populations (FIG. 3, Post-Cap). Both groups demonstrated a significant fall (p<0.001) in the HPT with an average of ˜7 C (see Table 2). Approximately 80% of the obese population (n=43/53) and 90% of the normal population (n=11/13; 2 complained of excessive pain before the heat probe could be applied) demonstrated a pronounced hyperalgesic response of 5° C.-16° C. Of 9 obese subjects who demonstrated a HPT at the peak temperature or did not report pain, all but one revealed a long latency/low amplitude blood flow response to acute capsaicin application.

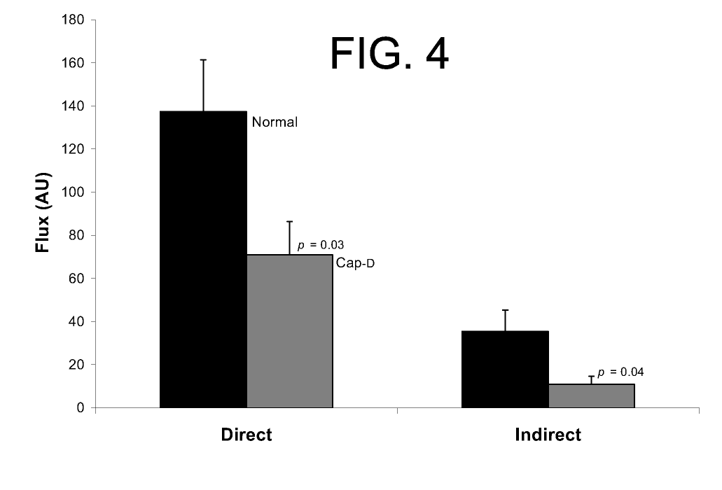

Responses to Heat-Step and Heat-Ramp Increases
1. Heat-Step Increases. Step increases in heat every 2° C. from 32° C. to 48° C. were associated with a significantly (p=0.007) increased HPT in the obese group (Table 2 and FIG. 3 Pre-Cap).
2. Heat-Ramp Increases. A heat-ramp stimulus from 32° C. to 44° C. (at an average speed of 2° C./sec.) caused a significant reduction in flare intensity of >50% (FIG. 4), whereas there was no significant difference between the two groups when comparing blood flow responses at the direct site (Table 2; FIG. 4). While the flare reactions were profoundly impaired, perceptual reports from obese subjects indicated that ˜45% claimed “pain/burn” sensation to or at 44° C., a value similar to that of the normal subjects. Approximately 15% (n=8) of the obese subjects had no change in sensation.

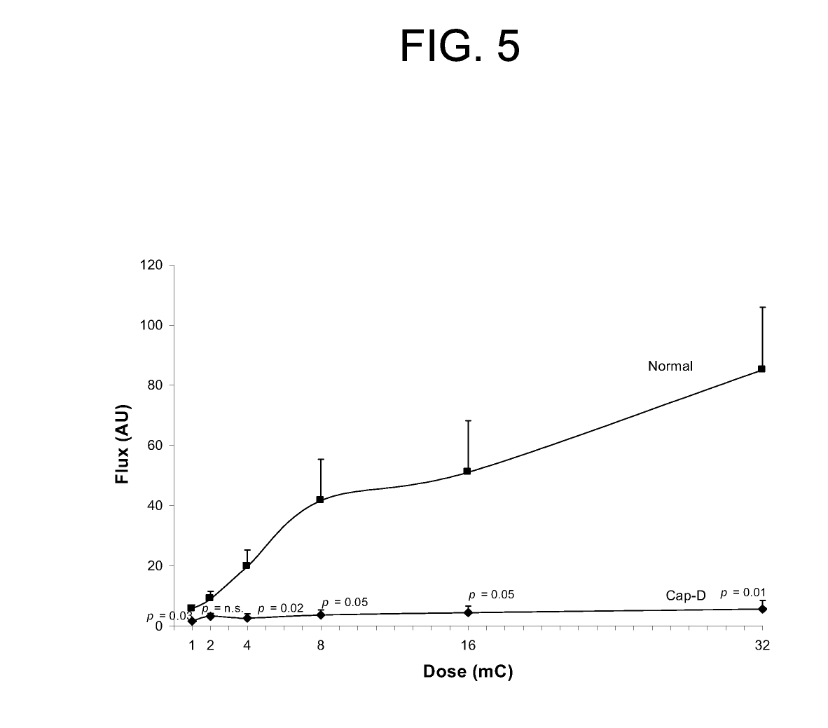

Dose-Response Relationships During Transcutaneous Electrical Stimulation: Dose (mC)-response (flux) curves were obtained during incremental increases in duration of stimulation at the same current intensity (0.2 mA). In both groups, direct and indirect flux increased as a function of current dose. Increasing charge or dose (mC) was associated with significantly lower flux at each dose in the obese group at both direct (not illustrated) and indirect sites (FIG. 5).

Correlations with BMI, Age, and Hyperglycemia: Pearson's correlation coefficients were used to examine the relationships between reported pain (in terms of threshold or presence of pungency) and flare with BMI, age, and hyperglycemia in each of the two groups. The only significant observation was that of age and capsaicin flare response (two-tailed, R²=−0.114; p=0.017) for the obese group. Age and capsaicin-induced flare for the normal group also indicated a negative correlation but the coefficient did not yield significant figures, most likely due to the small population sample. Using frequency histograms of Z scores for each or the sum of 6 sets of pain/flare data (i.e., HPT prior to and following capsaicin application, capsaicin pungency and flare latency and magnitude, and flare magnitude with electrical stimulation), the potential role of hyperglycemia and hyperinsulinemia was analyzed. Pearson correlations did not reveal significance between fasting hyperglycemia and normoglycemia (<100 mg/dl of glucose; <5.5 HbAlC) nor between hyperinsulinemia and normoinsulinemia.

Discussion: The application of capsaicin-sensitive tests (FIG. 1) to a large group of morbidly obese subjects reveals that small fiber, notably C- and Aδ-nociceptor, neuropathy (SSFN) is common in this population. The following observations are indicative of functional impairment of cutaneous nociceptor activation:
1. Decreased pungency, magnitude and latency of flare (FIG. 2A-C) to acute capsaicin treatment.
2. Raised HPT (FIG. 3) to heat-step and decreased flare to heat-ramp stimuli (FIG. 4).
3. Depressed flare (FIG. 5) to transcutaneous electrical stimulation.

Additionally, a number of issues are identified:
1. Approximately half of the obese subjects were neurologically asymptomatic with very few (n=4) reporting pain, an unanticipated outcome given the relatively high incidence of neuropathic pain in subjects with SSFN associated with IGT and diabetes.
2. Utilization of capsaicin-sensitive tests suggests that SSFN is a generalized disorder; in contrast, our clinical examination using heat/cold stimuli reveals a length-dependent or dying-back process, which may imply two distinct pathological processes.
3. The presence of hyperalgesia following capsaicin treatment in subjects with obvious SSFN suggests that the thermode size was sufficient to activate some nociceptors, including presumably “silent” nociceptors (e.g., mechanoinsensitive nociceptors), which would lead to summation of impulses by central neurons to cause pain (neurogenic hyperalgesia).
4. If depressed blood flow measured under the thermode (“direct flow”) during a heat-ramp test can be assumed to be an indicator of microangiopathy, obese subjects do not reveal a microangiopathic process (at least in the upper extremity) to account for the microcirculatory changes observed with capsaicin treatment and electrical stimulation.
5. Hyperglycemia (IGT, diabetes) is a potential contributing factor in SSFN among the obese subjects but only ˜70% were considered hyperglycemic (a 2-hour post-prandial test might prove more useful). A potential causative factor is oxidative stress which is raised in obesity, diabetes, insulin resistance, and neuropathy. Antioxidant therapy may improve nerve pathophysiology and, hence, reduce positive neuropathic sensory symptoms (e.g., pain).

Sir Thomas Lewis in 1937 postulated that nerves responsible for the axon-reflex (now identified as C- and Aδ-nociceptors) are associated with local defenses against injury (inflammation). He called these nerves “nocifensor nerves”, which implied that they elicited protective reactions such as hyperalgesia and flare responses to injury. Neurons participating in the local axon-reflex are considered peptidergic; the candidate peptide, thought to be released at the terminal endings of small nerve fibers, is CGRP.

Although a distinct system of nerves, namely nociceptors, generate signals which mediate acute and hyperalgesic pain and flare, pain and flare are not necessarily functionally coupled. For example, excitation of C-fiber discharges need not be associated with pain sensation but can evoke a flare response. The two may have different sensory/efferent pathways, and/or different sub-sets of nociceptors. The difference in the flare and pain response to a heat-ramp stimulus among the normal and obese subjects may be indicative of such a dissociation and, in fact, would favor a concept that the organism attempts to elicit an early protective reaction (neurogenic vasodilatation) at a temperature (44° C.) just below that which will become injurious to tissue.

Flare can be suppressed or abolished by denervation, local anesthetics, capsaicin-desensitization, disease (diabetes, post-herpetic neuralgia), and aging. Under circumstances of prolonged depression of small fiber function, tissue integrity can be substantially impaired. Animals and humans with suppressed C-fiber function are at high risk for wound occurrence and impaired wound healing. An intact nociceptor system of primary afferent neurons, e.g., capsaicin-sensitive nerves, is important in the initiation of the inflammatory process and successful tissue repair. Hence, our study suggests that obese subjects with/without diabetes would be at risk for impaired wound healing.

Indeed, the presence of a dysfunctional nociceptive system among obese subjects may be a critical factor in their often observed failure of wound healing. Although the obese subject may demonstrate a weakened flare reaction, sufficient blood flow may be attained (perhaps due to vascular receptor up-regulation) to ensure or enhance tissue integrity as revealed by observations of diabetic pressure ulcer improvement treated by transcutaneous electrical stimulation, TENS. Wound healing can also be enhanced by the exogenous application of peptides. CGRP has been used experimentally in animals and humans to promote enhanced rate of wound healing.

Age is an important determinant of the strength of nociceptive function. Aging animals demonstrate reduction in the number of C-fibers which is associated with decreased noxious thermal sensitivity and reduced flare responses. The latter reaction is particularly observed after chronic capsaicin treatment in young animals as well as among aged animals. In a sense, this connotes a premature depletion of nociceptive afferents in the young animals when treated with capsaicin, a most likely event in the obese population. In normal subjects, Helme recognized a negative correlation between capsaicin-induced flare magnitude and size with age. Similarly, both of our populations revealed a negative correlation between capsaicin-induced flare magnitude with age. Age-related loss of ENFs has also been reported. As an intact nociceptor system of primary afferent neurons with their sensory neuropeptides is an essential prerequisite for prevention of wounds and wound healing, the anticipated outcome would be increased prevalence of wounds and delay in healing among the aged population. Given that nociceptive function is likewise impaired in younger obese subjects, there would be a significant risk of failure to heal. Chang et al. report a higher complication rate for surgical flaps and donor sites in obese subjects, which is a similar outcome observed in animals after small sensory fiber denervation with capsaicin. Under these conditions, the rate of wound healing can be augmented by the application of TENS and/or exogenous peptides, such as CGRP, which are sufficient to evoke a flare reaction without a concomitant sensory response, i.e., pain perception.

### SUMMARY

In summary, we have observed that morbidly obese subjects demonstrate significant SSFN when challenged with tests considered to be capsaicin-sensitive and, hence, a measure of small primary afferent neuron function. This disorder is viewed as a generalized, largely asymptomatic process (despite a relatively high number of IGT and DM subjects) with a likely pathophysiologic mechanism concerned with oxidative stress. As in diabetes, SSFN in obesity mimics the aging process in which C-fibers are particularly vulnerable, but the generalization of the functional impairment varies with the clinical presentation (dying-back) suggesting two distinctly different pathological processes. Perhaps the latter is related to hyperglycemia with/without insulin resistance. The argument presented here is that SSFN observed in obesity is a similar process to that of age-related decrease in modulation of skin vascular reactivity by sensory nerves.

Future studies will address the relationship between small fiber function and density of epidermal nerve fibers, improvement of our diagnostic capacity for insulin resistance and DM/IGT, assessing the role of oxidative stress, and the study of the temporal pattern of development of somatic small fiber neuropathy. Whether SSFN should be considered an entity within the many factors comprising the metabolic syndrome or should be considered part of a continuum across factors needs to be determined, i.e., impairment of small fiber neuronal function may be viewed as a primary defect which can be inherited but is probably acquired, a contributing factor among the metabolic syndrome entities, or an epiphenomenon within the range of metabolic syndrome deficits.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. Any wound treatment with calcitonin gene-related peptide (CGRP) while monitoring blood flow proximal the wound.

2. The wound treatment as defined in claim 1, further comprising determining, while monitoring the blood flow, a dose/response relationship using CGRP.

3. The wound treatment as defined in claim 1, further comprising debriding the wound.

4. The wound treatment as defined in claim 1, wherein the wound is selected from the group consisting of a diabetic ulcer, a venous stasis ulcer, a sacral ulcer, a gluteal ulcer, a trochanteric ulcer, a blister ulcer, a varicose leg ulcer, a finger ulcer, and an ischemic skin flap.

5. The wound treatment as defined in claim 1, further comprising:
administering, with the CGRP, a vasodilator selected from the group consisting of:
vasoactive intestinal polypeptide (VIP);
nitric oxide (NO);
nerve growth factor (NGF); and
combinations of the forgoing; and
determining, from the monitoring, a dose/response relationship using CGRP and the vasodilator being administered.

6. The wound treatment as defined in claim 5, wherein the NO is a NO-donor.

7. The wound treatment as defined in claim 6, wherein the NO-donor can be sodium nitroprusside.

8. The wound treatment as defined in claim 5, further comprising supplementing the vasodilator with systemic L-Arginine and an anti-oxidant.

9. The wound treatment as defined in claim 8, wherein the anti-oxidant is alpha-lipoic acid.

10. The wound treatment as defined in claim 1, wherein the administration of the CGRP is at least one of active transdermal treatment and passive transdermal treatment.

11. The wound treatment as defined in claim 1, wherein the administration of the CGRP is selected from the group consisting of:
direct perfusion of the wound with the CGRP;
an intradermal injection of the CGRP about the wound and its surrounding skin area;
iontophoresis about the wound and its surrounding skin area with the CGRP;
microdialysis of the wound with the CGRP using one or more probe situated about the wound and its surrounding skin area;
transcutaneous electrical stimulation (TENS) about the wound and its surrounding skin area while administering the CGRP; and
a combination of the foregoing treatments.

12. The wound treatment as defined in claim 1, wherein the wound is a wound in the skin of a mammal.

13. A method comprising administering a vasodilator to a wound in a treatment that is at least one of active transdermal treatment and passive transdermal treatment.

14. The method as defined in claim 13, wherein the treatment selected from the group consisting of:
direct perfusion of the wound with the vasodilator;
an intradermal injection of the vasodilator about the wound and its surrounding skin area;
iontophoresis about the wound and its surrounding skin area with the vasodilator;
microdialysis of the wound with the vasodilator using one or more probe situated about the wound and its surrounding skin area;
transcutaneous electrical stimulation (TENS) about the wound and its surrounding skin area while administering the vasodilator; and
a combination of the foregoing treatments.

15. The method as defined in claim 13, wherein the vasodilator is selected from the group consisting of:
calcitonin gene-related peptide (CGRP);
vasoactive intestinal polypeptide (VIP);
nitric oxide (NO);
nerve growth factor (NGF); and
combinations of the forgoing.

16. The method as defined in claim 15, wherein the NO is a NO-donor.

17. The method as defined in claim 16, wherein the NO-donor can be sodium nitroprusside.

18. The method as defined in claim 15, further comprising supplementing the vasodilator with systemic L-Arginine and an anti-oxidant.

19. The method as defined in claim 18, wherein the anti-oxidant is alpha-lipoic acid.

20. The method as defined in claim 13, wherein said administering further comprises monitoring blood flow proximal the wound.

21. The method as defined in claim 20, wherein the monitoring of the blood flow is accomplished by a technique selected from the group consisting of laser Doppler flowmetry (LDF), laser Doppler perfusion imagery (LDPI), and a combination thereof.

22. The method as defined in claim 20, wherein said monitoring further comprises determining, from said monitoring, a dose/response relationship of the vasodilator being administered in the treatment.

23. The method as defined in claim 13, further comprising, prior to said treatment, debriding the wound.

24. The method as defined in claim 13, wherein the wound is selected from the group consisting of a diabetic ulcer, a venous stasis ulcer, a sacral ulcer, a gluteal ulcer, a trochanteric ulcer, a blister ulcer, a varicose leg ulcer, a finger ulcer and an ischemic skin flap.

25. A method comprising administering of treatment of calcitonin gene-related peptide (CGRP) to a wound while monitoring blood flow proximal the wound, wherein the treatment is at least one of active transdermal treatment and passive transdermal treatment.

26. The method as defined in claim 25, wherein the treatment selected from the group consisting of:
direct perfusion of the wound with the CGRP;
an intradermal injection of the CGRP about the wound and its surrounding skin area;
iontophoresis about the wound and its surrounding skin area with the CGRP;
microdialysis of the wound with the CGRP using one or more probe situated about the wound and its surrounding skin area;
transcutaneous electrical stimulation (TENS) about the wound and its surrounding skin area while administering the CGRP and
a combination of the foregoing treatments.

27. The method as defined in claim 25, wherein the CGRP is also administered with a vasodilator selected from the group consisting of:
vasoactive intestinal polypeptide (VIP);
nitric oxide (NO);
nerve growth factor (NGF); and
combinations of the forgoing.

28. The method as defined in claim 27, wherein the NO is a NO-donor.

29. The method as defined in claim 28, wherein the NO-donor can be sodium nitroprusside.

30. The method as defined in claim 27, further comprising supplementing the vasodilator with systemic L-Arginine and an anti-oxidant.

31. The method as defined in claim 30, wherein the anti-oxidant is alpha-lipoic acid.

32. The method as defined in claim 25, wherein the monitoring of the blood flow is accomplished by a technique selected from the group consisting of laser Doppler flowmetry (LDF), laser Doppler perfusion imagery (LDPI), and a combination thereof.

33. The method as defined in claim 25, wherein said monitoring further comprises determining, from said monitoring, a dose/response relationship of CGRP being administered in the treatment.

34. The method as defined in claim 27, wherein said monitoring further comprises determining, from said monitoring, a dose/response relationship using CGRP and the vasodilator being administered in the treatment.

35. A method comprising:
debriding a wound selected from the group consisting of a diabetic ulcer, a venous stasis ulcer, a sacral ulcer, a gluteal ulcer, a trochanteric ulcer, a blister ulcer, a varicose leg ulcer, a finger ulcer, and an ischemic skin flap;
administering to the debrided wound, while monitoring blood flow proximal thereto, a vasodilator selected from the group consisting of:
calcitonin gene-related peptide (CGRP);
vasoactive intestinal polypeptide (VIP);
nitric oxide (NO);
nerve growth factor (NGF); and
combinations of the forgoing; and
determining, from the monitoring, a chemical/dose relationship of the vasodilator being administered.

36. The method as defined in claim 35, wherein the vasodilator is administered to the wound by at least one of active transdermal treatment and passive transdermal treatment.

37. The method as defined in claim 35, wherein the vasodilator is administered to the wound by a treatment that is selected from the group consisting of:
direct perfusion of the wound with the vasodilator;
an intradermal injection of the vasodilator about the wound and its surrounding skin area;
iontophoresis about the wound and its surrounding skin area with the vasodilator;
microdialysis of the wound with the vasodilator using one or more probe situated about the wound and its surrounding skin area;
transcutaneous electrical stimulation (TENS) about the wound and its surrounding skin area while administering the vasodilator; and
a combination of the foregoing treatments.
